(19) United States
(12) Patent Application Publication
(10) Pub. No.: US 2025/0032501 A1
(43) Pub. Date: Jan. 30, 2025

(54) METHODS AND COMPOSITIONS FOR THE TREATMENT OF ALZHEIMER’S DISEASE

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Frederic Geissmann, New York, NY (US); Rocio Vicario, New York, NY (US)

(21) Appl. No.: 18/580,831

(22) PCT Filed: Jul. 21, 2022
(86) PCT No.: PCT/US2022/037893

§ 371 Date: Jan. 19, 2024

Related U.S. Application Data
(60) Provisional application No. 63/225,056, filed on Jul. 23, 2021.

(51) Int. Cl. A61K 31/5377; A61K 31/166; A61K 31/18; A61K 31/365; A61K 31/404; A61K 31/4184; A61K 31/437; A61K 31/44; A61K 31/4412; A61K 31/4418; A61K 31/4439; A61K 31/4523; A61K 31/4709; A61K 31/496; A61K 31/505; A61K 31/506; A61K 31/517; A61K 31/519; A61P 25/28

(52) U.S. Cl. CPC A61K 31/5377; A61K 31/166; A61K 31/18; A61K 31/365; A61K 31/404; A61K 31/4184; A61K 31/437; A61K 31/44; A61K 31/4412; A61K 31/4418; A61K 31/4439; A61K 31/4523; A61K 31/4709; A61K 31/496; A61K 31/505; A61K 31/506; A61K 31/517; A61K 31/519; A61P 25/28

(57) ABSTRACT

The present technology relates to methods for treating, preventing, and/or ameliorating Alzheimer's disease, in a subject in need thereof. In particular aspects, the present technology relates to the use of MAPK inhibitors to treat, prevent, and/or ameliorate Alzheimer's disease.

### CROSS-REFERENCE TO RELATED APPLICATIONS

This application claims the benefit of and priority to U.S. Provisional Patent Application No. 63/225,056, filed Jul. 23, 2021, the entire contents of which are incorporated herein by reference.

### STATEMENT OF RIGHTS UNDER FEDERALLY-SPONSORED RESEARCH

This invention was made with government support under CA008748, NS 115715, HL138090, and AI130345 awarded by the National Institutes of Health. The government has certain rights in the invention.

### TECHNICAL FIELD

The present disclosure relates to a method for treatment of Alzheimer's disease.

### BACKGROUND

Alzheimer's disease (AD) is a progressive neurodegenerative disorder of the brain, which is characterized by the memory deterioration, behavioral disturbances, impairment of activities of daily living, and loss of independent function. Alzheimer's disease is the most frequent cause of progressive dementia and its prevalence increases with age, with an estimated prevalence of 10% after 65 and 30% after 85. Environmental factors and co-morbidities play important roles in the development of AD. The role of germline genetic variation in AD has been studied intensely. Autosomal dominant AD, due to germline mutations in presenilin 1 (PSEN1), presenilin 2 (PSEN2), and amyloid precursor protein (APP) account for an estimated 1% of patients. Carriers of one germline copy of the epsilon4 (ε4) allele of the apolipoprotein E gene (APOE4) have a three-fold higher risk of AD, and two copies increase AD risk ˜10-fold. Genome-wide association studies (GWAS) have identified an additional ˜50 genetic variants that more moderately increase the risk of AD including, TREM2, CD33, and MS4A6A variants. Clonal heterogeneity (mosaicism) is widespread in human tissues and causes tumoral, developmental, and immune diseases, and mosaicism in the human brain can cause diseases. The contribution of somatic clones, resulting from post-zygotic somatic DNA mutations, has been the subject of few studies in neurodegeneration.

The sensitivity of these early sequencing studies, performed in whole brain, has also been limited by the sequencing depth. In addition, a growing body of evidence indicating a heterogeneity of AD, coupled with disappointing clinical studies of fit-for-all therapies, suggests that a more tailored or personalized treatment is required for specific AD subpopulations sharing genetic or pathological properties. Accordingly, there is a need to develop deep sequencing studies to identify additional targets for the treatment and prevention of AD.

### SUMMARY

The technology of the present disclosure relates to the observation that, through deep sequencing of blood and brain samples, microglial clones from humans with Alzheimer's disease (AD) were more likely to carry pathogenic mutations as compared to age-matched controls, that the pathogenic microglia frequently carried mutations that activate the MAP kinase (MAPK) pathway (p:0.03 as compared to age-matched controls), and that the AD subjects carrying the pathogenic microglia are less likely to carry the apolipoprotein E (APOE) epsilon4 (ε4) risk allele (APOE4; p: 0.02). As demonstrated herein, pathogenic microglial clones frequently carry mutations that activate the MAPK pathway, including recurrent loss-of-function mutations in the ring-type domain of CBL, and microglia from AD patients display in situ ERK phosphorylation. Accordingly, microglial clones, in particular those with dysregulated MAPK activation, may contribute to the pathogenesis of AD, at least among patients with low APOE4 genetic risk, and can serve as a target for a more personalized and efficient approach for diagnosing and treating AD subpopulations that share these genetic or pathological properties.

In one aspect, the present disclosure provides a method for treating or preventing Alzheimer's disease in a subject in need thereof, comprising administering to the subject a therapeutically effective amount of a MAP kinase (MAPK) inhibitor, or a pharmaceutically acceptable salt thereof, wherein the subject is suspected of having, at risk for, or diagnosed as having Alzheimer's disease.

In another aspect, the present disclosure provides a method for delaying the onset of one or more signs or symptoms of Alzheimer's disease in a subject in need thereof, comprising administering to the subject a therapeutically effective amount of a MAP kinase (MAPK) inhibitor, or a pharmaceutically acceptable salt thereof, wherein the subject is suspected of having, at risk for, or diagnosed as having Alzheimer's disease.

In another aspect, the present disclosure provides a method for increasing the survival of a subject in need thereof, comprising administering to the subject a therapeutically effective amount of a MAP kinase (MAPK) inhibitor, or a pharmaceutically acceptable salt thereof, wherein the subject is suspected of having, at risk for, or diagnosed as having Alzheimer's disease.

In another aspect, the present disclosure provides a method for attenuating cognitive decline in a subject in need thereof, comprising administering to the subject a therapeutically effective amount of a MAP kinase (MAPK) inhibitor, or a pharmaceutically acceptable salt thereof, wherein the subject is suspected of having, at risk for, or diagnosed as having Alzheimer's disease.

In another aspect, the present disclosure provides a method for treating or preventing Alzheimer's disease, delaying the onset of one or more signs or symptoms of Alzheimer's disease, increasing the survival of a subject, and/or attenuating cognitive decline in a subject in need thereof, wherein the subject is suspected of having, at risk for, or diagnosed as having Alzheimer's disease, the method comprising: (a) isolating a tissue sample from the subject; (b) determining whether the tissue expresses a MAPK-activating mutation; and (c) administering to the subject a therapeutically effective amount of a MAPK inhibitor, or a pharmaceutically acceptable salt thereof, when the isolated tissue expresses the MAPK-activating mutation.

In some embodiments, the tissue sample is blood or brain tissue.

In some embodiments, the MAPK-activating mutation comprises a mutation in one or more genes selected from CBL, RIT1, BRAF, PTPN11, KRAS, NF1, and FAM58A.

In some embodiments, the mutation in CBL, NF1, or FAM58A is a loss-of-function mutation, and wherein the mutation in RIT1, BRAF, KRAS, or PTPN11 is a gain-of-function mutation.

In some embodiments, the CBL loss-of-function mutation comprises one or more of 1383M, C404Y, C416S, C384Y, and R420Q; the NF1 loss-of-function mutation comprises c.7325T>G STOP; the RIT1 gain-of-function mutation comprises one or more of F99C and M107V; the BRAF gain-of-function mutation comprises L505H; the KRAS gain-of-function mutation comprises A59G; the PTPN11 gain-of-function mutation comprises T73I; and the FAM58A loss-of-function mutation comprises c.423+5A>C.

In some embodiments, the mutation comprises the CBL C404Y mutation.

In some embodiments, the mutation comprises the CBL C416S mutation.

In some embodiments, the mutation comprises the CBL C384Y mutation.

In some embodiments, the mutation comprises the RIT1 F99C mutation.

In some embodiments, the mutation comprises the RIT1 M107V mutation.

In some embodiments, the subject comprises a MAPK-activating mutation in one or more genes selected from CBL, RIT1, BRAF, PTPN11, KRAS, NF1, and FAM58A.

In some embodiments, the mutation in CBL, NF1, or FAM58A is a loss-of-function mutation, and wherein the mutation in RIT1, BRAF, KRAS, or PTPN11 is a gain-of-function mutation.

In some embodiments, the CBL loss-of-function mutation comprises one or more of 1383M, C404Y, C416S, C384Y, and R420Q; the NF1 loss-of-function mutation comprises c.7325T>G STOP; the RIT1 gain-of-function mutation comprises one or more of F99C and M107V; the BRAF gain-of-function mutation comprises L505H; the KRAS gain-of-function mutation comprises A59G; the PTPN11 gain-of-function mutation comprises T73I; and the FAM58A loss-of-function mutation comprises c.423+5A>C.

In some embodiments, the mutation comprises the CBL C404Y mutation.

In some embodiments, the mutation comprises the CBL C416S mutation.

In some embodiments, the mutation comprises the CBL C384Y mutation.

In some embodiments, the mutation comprises the RIT1 F99C mutation.

In some embodiments, the mutation comprises the RIT1 M107V mutation.

In some embodiments, the subject is a non-carrier for APOE4 risk alleles.

In some embodiments, the subject carries one APOE4 risk allele.

In some embodiments, the subject is suspected of having, is at risk for, or is diagnosed as having intermediate onset Alzheimer's disease.

In some embodiments, the Alzheimer's disease is characterized by one or more of: cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders.

In some embodiments, the MAPK inhibitor is selected from the group consisting of: Gefitinib, Erlotinib, Lapatinib, Sunitinib, Sorafenib, Nilotinib, Dasatinib, Imatinib, Tipifarnib, Sorafenib, U0126, PD184352, AZD6244, BIX02188, BIX02189, SB203580, SB202190, BIRB-796, LY2228820, VX-702, VX-745, and TAK-715.

In some embodiments, the route of administration of the MAPK inhibitor is parenteral, intradermal, intramuscular, intraperitoneal, intravenous, subcutaneous, intranasal, epidural, oral, sublingual, intranasal, intracerebral, intracerebroventricular, intrathecal, intravaginal, transdermal, rectal, by inhalation, or topical.

In some embodiments, the methods further comprise separately, sequentially, or simultaneously administering an additional therapeutic agent to the subject.

In some embodiments, the additional therapeutic agent comprises a BRAF, MEK, and/or CSF-1R inhibitor, or a pharmaceutically acceptable salt thereof.

In some embodiments, the BRAF inhibitor is selected from the group consisting of vemurafenib, dabrafenib, encorafenib, PLX7904, PLX8394, GDC-0879, LGX818, and PLX4720, the MEK inhibitor is selected from the group consisting of AZD8330, refametinib, E6201, MEK162 (binimetinib), PD0325901, pimasertib, R04987655, selumetinib, TAK-733, GDC-0623, WX-544, cobimetinib, and trametinib, and the CSF-1R inhibitor is selected from the group consisting of PLX5622, GW2580, BLZ945, pexidartinib (PLX3397), ARRY-382, PLX7486, and JNJ-40346527.

In some embodiments, the combination of MAPK inhibitor and the additional therapeutic agent has a synergistic therapeutic effect.

In some embodiments, the subject is human.

### BRIEF DESCRIPTION OF THE DRAWINGS

FIG. 1A shows a schematic representation of purification of nuclei from PU.1⁺ (myeloid/microglia), NeuN⁺ (neurons), and double negative glial cells (DN) by flow cytometry, and a histogram representing the relative frequency of nuclei for each cell types in controls (n=63 samples) and patients (n=99 samples).

FIG. 1B shows deep-sequencing, analysis, and validation pipeline.

FIG. 1C shows frequency of variants per Mb among cell types (NeuN: n=201, DN: n=204, PU.1: n=201, blood: n=58), mean number of mutations/Mb is indicated in italics, p-value are calculated by one-way ANOVA and Tukey test for multiple comparisons.

FIG. 1D shows a histogram representing the frequency of variants per Mb from FIG. 1C among disease groups.

FIG. 1E shows Venn diagrams representing the repartition of the 692 variants per cell types and patient group. Boxes indicate genes targeted by mutations shared between blood and PU.1⁺ cells.

FIG. 2A shows top10 missense mutations predicted deleterious by 4 predictors (Polyphem and SIFT and CADD/MSC and COSMIC) in neurons (NeuN⁺), glia (DN), microglia (PU.1⁺) and blood, and (%) of samples carrying these mutations in controls (>55).

FIG. 2B shows top10 missense mutations predicted deleterious by 4 predictors (Polyphem and SIFT and CADD/MSC and COSMIC) in neurons (NeuN⁺), glia (DN), microglia (PU.1⁺) and blood, and (%) of samples carrying these mutations in AD patients.

FIG. 2C shows the number of mutations reported to be pathogenic by ClinVar and OncoKB in neurons (NeuN⁺), glia (DN), microglia (PU.1⁺) and blood, per Mb and per sample. p-values are calculated with a unpaired two-tailed Mann-Whitney U test.

FIG. 2D shows the number of mutations reported to be pathogenic by ClinVar and OncoKB in microglia (PU.1⁺). p-values are calculated with a unpaired two-tailed Mann-Whitney U test. Percentage of patients carrying mutations is shown in (italics) and p-value for comparison with age-matched controls is calculated using two-sided Fisher's exact test.

FIG. 2E shows genes targeted by pathogenic mutations in PU.1⁺ cells from FIGS. 2C/2D: left axis, bars indicate the number of patients carrying mutations; right x axis, circles indicate allelic frequency of the variants. When a mutation is present in 2 PU.1⁺ samples from the same patients, both AF are indicated. Only mutation reported to be pathogenic (from FIGS. 2C/2D) are plotted.

FIG. 2F shows Left (*): distribution of APOE genotype in patients and control from a reference study by Sando, S. B., et al., BMC Neurol 8: 9 (2008), doi:10.1186/1471-2377-8-9; Right: distribution of all AD patients from this study by APOE genotype and the presence of pathogenic and/or predicted pathogenic mutations in PU.1⁺ cells. p-value is calculated using two-sided Fisher's exact test.

FIG. 3 shows the characteristics of mutations that disrupt protein function (pathogenic) in AD patients. Mutations characterized in this study are marked with $. Lines indicate the patient ID number, the number of brain regions harboring the mutant clone, consequences (GOF or LOF) of the mutation on gene function, the detection of the mutation in the blood of corresponding patient, other mutations detected in PU.1⁺ cells from the same patient, APOE genotype of the patient, and the pathways assigned to the mutations proposed by the inventors.

FIG. 4A shows the CBL structure. The positions of the microglia-associated mutations are shown in red on the 3D structure (pdb code: 1fbv).

FIG. 4B shows expressions of CBL in HEK293T cells. Cell lysates were probed with antibodies against phospho-MAPK and total MAPK.

FIG. 4C shows the structure (Left) and cell expression (Right) of RIT1. The position of M107 is shown in red on the 3D structure (pdb code: 4klz). F99 is within a segment whose structure was not resolved. Flag-RIT1 was expressed in HEK293T cells. Cells were treated −/+20% FBS before harvesting. Lysates were probed with antibodies against phospho-MAPK, total MAPK, and Flag.

FIG. 4D shows the classical MAPK pathway with genes targeted by pathogenic mutations identified in this work, which are underlined and shown in italics. FAM58A is underlined but not italicized, as it is not included in the classical MAPK genes.

FIG. 4E shows the number of mutations in classical MAPK-pathway genes (mutation/sample/Mb) in microglia (PU.1⁺) of AD and age-matched controls. p-values are calculated with a unpaired two-tailed Mann-Whitney U test. Percentage of patients carrying mutations is shown in italics.

FIG. 4F shows the expression of pERK in microglia from brains without AD lesions (ctrl) or with AD (AD), brains of patients with ECDs are used as positive control.

FIG. 4G shows the domain structure of CHEK2 with the positions of mutations in patients AD23 and AD57 indicated, and CHEK2 expression in HEK293T cells. R346 is shown in red on the 3D structure of CHEK2 kinase domain (pdb code: 2cn5). Flag-CHEK2 was expressed in HEK293T cells. Lysates were probed with antibodies against phospho-CHEK2 and Flag. Bottom: CHEK2 was immunoprecipitated from 293T cells and assayed with a synthetic peptide substrate.

FIG. 4H shows hypothetic model illustrating how clones of dysfunctional microglial carrying driver mutations may expand in patient brains over time and disrupt neuronal homeostasis and the fitness of neuronal networks, facilitating neuronal death and circuit dysfunction and contributing to clinical dementia.

FIG. 5A shows the number of samples sequenced in this study in each group.

FIG. 5B shows the sorting strategy used to separate PU.1⁺, NEUN⁺ and DN nuclei from post-mortem brain samples. Sorting purity was >90%.

FIG. 5C shows validation of mutation calls. Plots comparing the VAF by targeted-sequencing and by ddPCR on 63 validated variants (˜10% of total events, ˜90% validity). The fitted line, R² value, and P value were obtained by linear regression.

FIG. 6A shows (Left) missense mutations predicted deleterious by 4 predictors (Polyphem and SIFT and CADD/MSC and COSMIC) in (PU.1⁺) from AD and age-matched controls (p-values are calculated with a unpaired two-tailed Mann-Whitney U test), and (Right) representation of the proportion of patients with the most recurrent (top10) mutations predicted deleterious.

FIG. 6B shows Voom normalized expression in whole brain and microglia (M) of genes carrying mutations found in PU.1 cell and reported to be pathogenic in human diseases by ClinVar and OncoKB. Data plotted from Galatro et al., [doi: 10.1038/nn.4597, ncbi.nlm.nih.gov/geo/query/acc.cgi?acc=GSE99074]. Sorted microglia n=39, and whole brain n=16.

FIG. 6C shows Deseq2 normalized expression in whole brain and microglia (M) of genes carrying mutations found in PU.1 cell and reported to be pathogenic in human diseases by ClinVar and OncoKB. Data plotted from Gosselin D., et al., Science 23: eaa13222 [DOI: 10.1126/science.aa13222]. Sorted microglia n=3 and whole brain n=1.

FIG. 7 shows pulldown experiments with wild-type and mutant forms of RIT1. Flag-tagged RIT1 constructs were expressed in 293T cells. Lysates were used in pulldown reactions with immobilized GST-PAK1-CRIB domain and immunoprecipitation reactions with Cdc42 antibody. Bound RIT1 was measured by anti-Flag Western blotting. Lysates were also analyzed by anti-Flag and anti-MAPK Western blotting.

### DETAILED DESCRIPTION

All patents, published applications and other publications and references are hereby incorporated by reference in their entirety into the present disclosure.

It is to be appreciated that certain aspects, modes, embodiments, variations and features of the present technology are described below in various levels of detail in order to provide a substantial understanding of the present technology.

### Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a,” “an,” and “the” include plural referents unless the content clearly dictates otherwise. For example, reference to “a cell” includes a combination of two or more cells, and the like.

As used herein, the term “about” encompasses the range of experimental error that may occur in a measurement and will be clear to the skilled artisan.

As used herein, the term “MAP kinase-activating mutation,” or “MAPK-activating mutation” refers to a mutation in one or more genes selected from CBL, RIT1, BRAF, PTPN11, KRAS, NF1, and FAM58A. In some embodiments, the mutation in CBL, NF1, or FAM58A is a loss-of-function mutation, and the mutation in RIT1, BRAF, KRAS, or PTPN11 is a gain-of-function mutation. In some embodiments, the CBL loss-of-function mutation comprises one or more of 1383M, C404Y, C416S, C384Y, and R420Q; the NF1 loss-of-function mutation comprises c.7325T>G STOP; the RIT1 gain-of-function mutation comprises one or more of F99C and M107V; the BRAF gain-of-function mutation comprises L505H; the KRAS gain-of-function mutation comprises A59G; the PTPN11 gain-of-function mutation comprises T731; and the FAM58A loss-of-function mutation comprises c.423+5A>C.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the terms “subject,” “individual,” or “patient” can be an individual organism, a vertebrate, a mammal, or a human.

As used herein, a “therapeutically effective amount” of a compound refers to compound or agent levels in which the physiological effects of a disease or disorder are, at a minimum, ameliorated. A therapeutically effective amount can be given in one or more administrations.

The amount of a compound which constitutes a therapeutically effective amount will vary depending on the compound, the disorder and its severity, and the general health, age, sex, body weight and tolerance to drugs of the subject to be treated, but can be determined routinely by one of ordinary skill in the art.

“Treating,” “treat,” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

The term “administer,” “administering,” or “administration” as used in this disclosure refers to either directly administering a therapeutic agent, such as, but not limited to, a MAPK inhibitor to a subject.

In certain embodiments, routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In some embodiments, the administering is effected orally or by parenteral injection. The mode of administration can be left to the discretion of the practitioner, and will depend in-part upon the site of the medical condition. In most instances, administration results in the release of any agent described herein into the bloodstream. In specific embodiments, it may be desirable to administer locally to the area in need of treatment or blocking.

### General

Microglia dysfunction has been implicated in the pathogenesis of AD, and microglial clones carrying a mutation activating the MAP kinase (MAPK) pathway cause neurodegeneration in mice, but whether a clonal process may underlie AD pathogenesis has not been investigated. The inventors of the present technology carried out deep-sequencing of neurons, glia, microglia, and matching blood DNA from a series of control and intermediate-onset sporadic AD patients (mean age at death 65). The present technology relates to the discovery that microglia carry a greater clonal diversity than neurons and glia, and somatic clones carrying pathogenic driver-mutations (somatic mutations that confer a proliferative, survival, or activation advantage (called “oncogenic” or “driver” mutations)) are found in large excess in the microglia from AD patients in comparison to age-matched controls (p:0.0005). Moreover, as noted above and described in more detail in the experimental examples, among AD patients, those carrying microglial clones are less likely to carry the apolipoprotein E4 risk allele (APOE4). In addition, as demonstrated herein, pathogenic microglial clones frequently carry mutations that activate the MAPK pathway (p:0.03 in comparison to age-matched controls), including recurrent loss-of-function mutations in the ring-type domain of CBL, and microglia from AD patients display in situ ERK phosphorylation.

Accordingly, microglial clones, in particular those with dysregulated MAPK activation, may contribute to the pathogenesis of AD, at least among patients with low APOE4 genetic risk, and can serve as a target for a more personalized and efficient approach for diagnosing and treating AD subpopulations that share these genetic or pathological properties. Due to the heterogeneous nature of AD, there is a need for the development of more personalized therapeutic and diagnostic approaches for AD. The methods and compositions of the present technology therefore provide a more personalized, targeted therapeutic approach for a subpopulation of subjects diagnosed with, at risk for, or suspected of having AD.

As described in more detail in the experimental examples, the inventors of the present technology have shown that PU.1 Vmicroglia carry a greater clonal diversity than neurons and glia, and that brains of AD patients are characterized by a large excess of microglia PU.1⁺ clones carrying pathogenic driver mutations. Among these, a fraction correspond to clonal hematopoiesis (CH) mutations, mainly TET2 and DNMT3, shared between circulating blood and brain PU.1. In the absence of epidemiological evidence, it cannot be ruled out that the presence of blood clones in the brain of AD patients could be a secondary event associated with leucocyte recruitment to a diseased brain. In addition, and sometimes in the same patients, private brain clones, which are not detected in the patients' matching blood, harbor pathogenic mutations that activate MAP kinase signaling in 24% of the patients in this series, including recurrent CBL mutations in 6 patients, or inactivate tumor suppressors. These private brain clones likely represent mutant resident microglia, consistent with previous findings that microglia are largely independent from bone marrow hematopoiesis for their maintenance in adults.

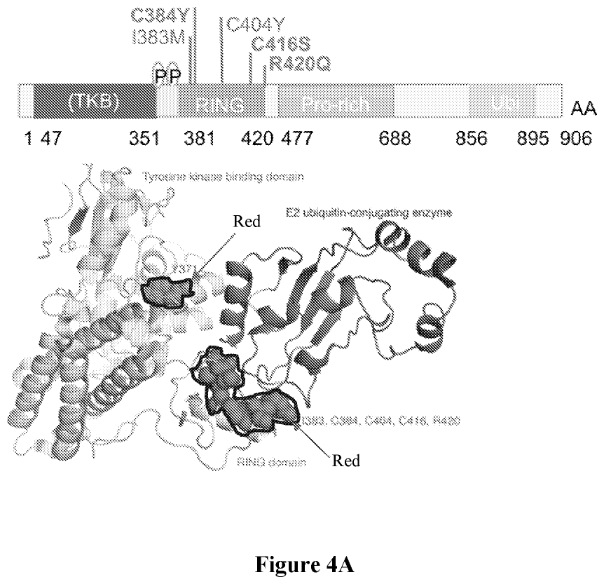

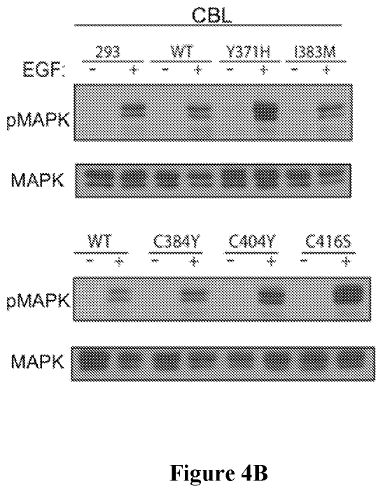

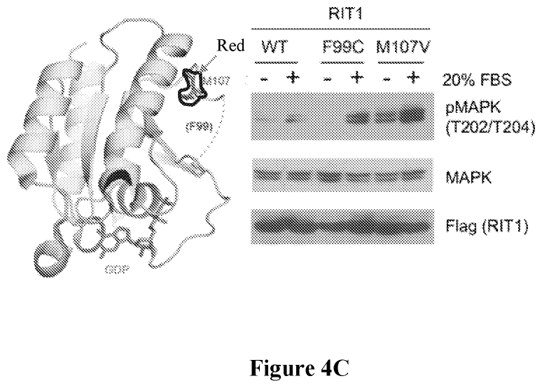

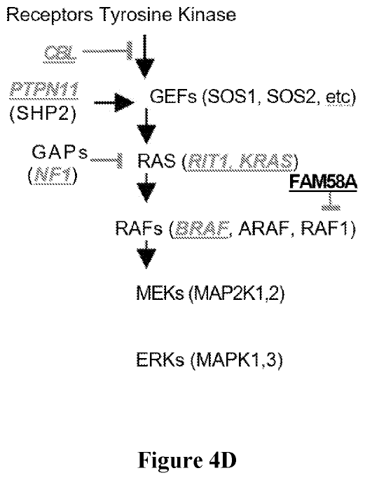

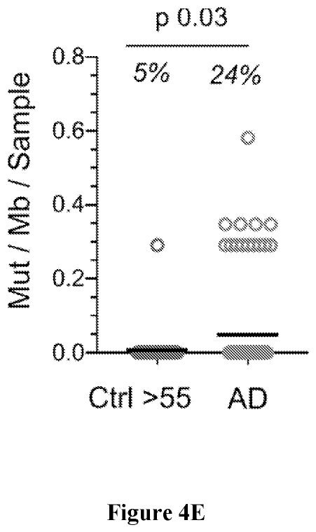

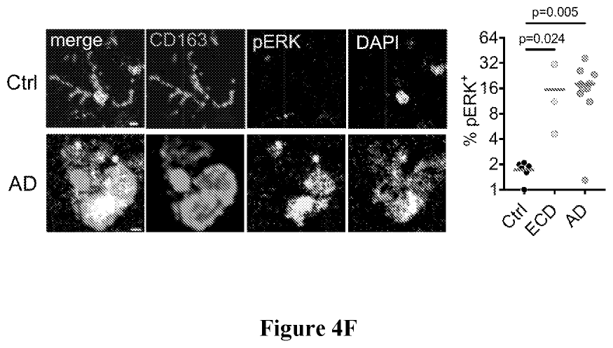

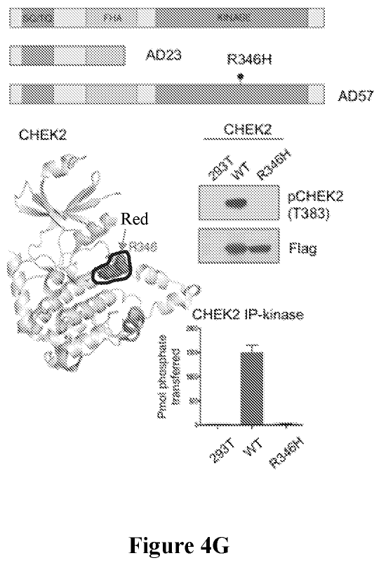

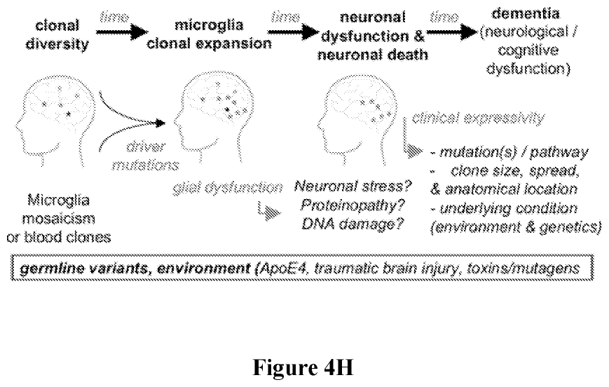

The data presented herein provide evidence in support of the hypothesis that these mutant microglia may contribute to the onset or progression of AD. As noted above, patients carrying microglial clones are less likely to carry the APOE4 risk allele, ERK activation is a feature of macrophages in AD patients' tissues, and mutant clones are found in the hippocampus. Furthermore, microglia clones carrying another MAPK activating mutation (e.g., BRAFV^{600E}) cause neurodegeneration in mice and likely in human histiocytosis patients at comparable allelic frequencies. Moreover, germline CBL, RIT1, and NF1 MAP kinase activating mutations in Rasopathies, as well as germline tumor suppressors mutations, are associated with cognitive impairment and disrupt CNS development in a mutation specific manner. Given strong germline genetic evidence that microglial dysfunction is involved in AD pathogenesis, it is therefore possible to speculate that microglial clones carrying driver mutations, specifically activating the MAPK pathway, expand in patient brains over time and that these dysfunctional microglia disrupt neuronal homeostasis and the fitness of neuronal networks, facilitating neuronal death and circuit dysfunction and contributing to clinical dementia (FIG. 4H).

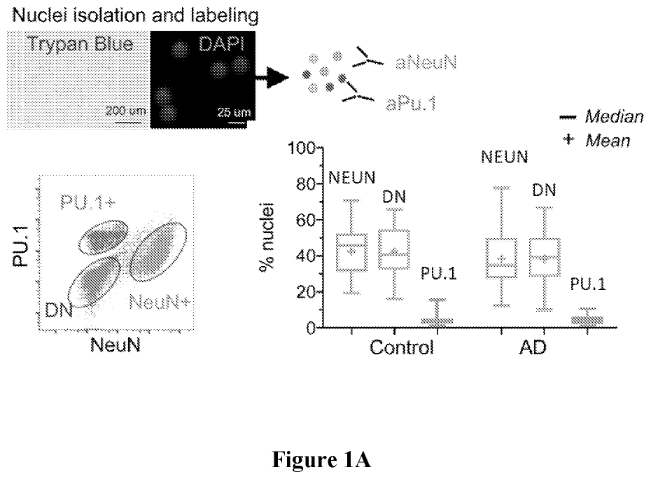

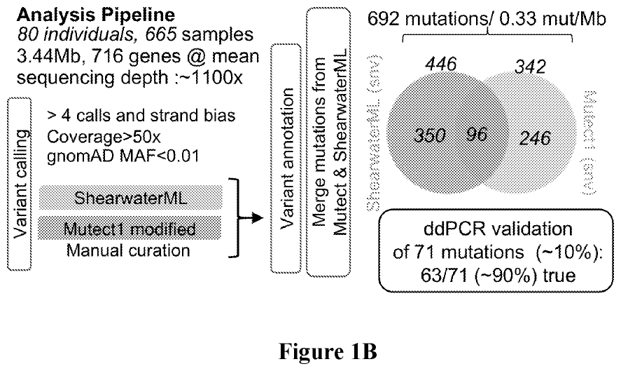

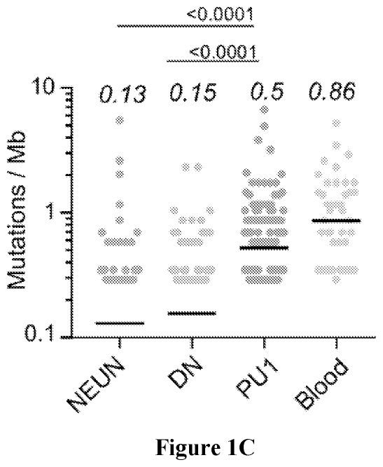

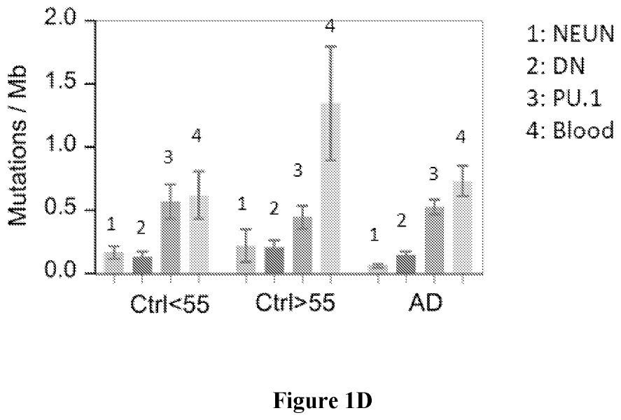

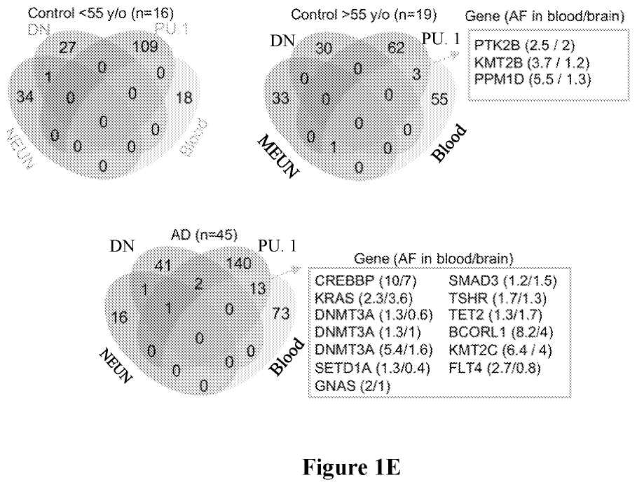

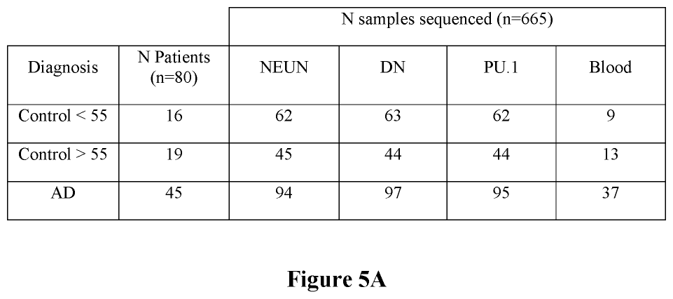

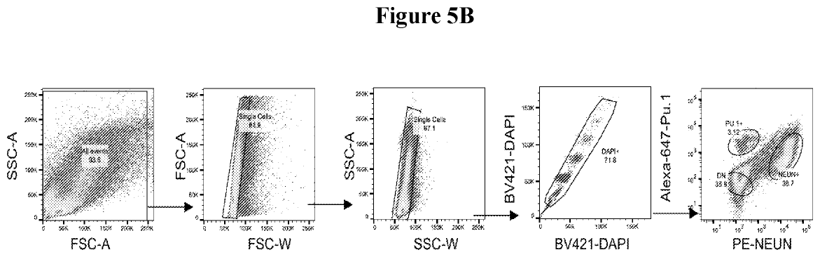

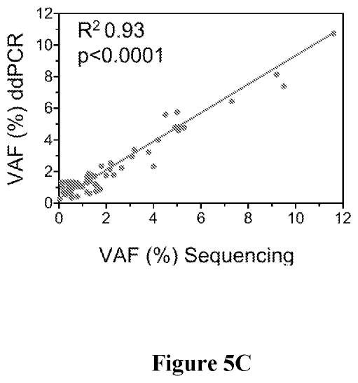

The inventors of the present technology tested the general hypothesis that driver mutations conferring proliferative/survival/activation advantage to dominant clones may cause or contribute to neuronal damage in AD patients by investigating the presence of somatic clones in the patient's brain at increased depth and cellular resolution. The brain is composed of several specialized cell types, and it has been noted that APOE, as well as two-thirds of the germline AD-risk SNPs are genes most highly expressed in macrophages, with variants largely confined to microglia enhancers, supporting a long-held hypothesis that microglia, the brain macrophages, may play an important role in the pathogenesis of AD although their possible pathogenic or protective role(s) has been difficult to resolve in genetic or molecular terms. Because microglia only represent an average ˜5% of brain cells while other glia and neurons represent ˜40-50% each (FIG. 1A), brain cell types were separated prior to deep sequencing to increase sensitivity, each cell type was analyzed at the same sequencing depth, and cells carrying deleterious mutants were identified (FIGS. 1A-1B, FIG. 5A). The human microglia is formed without meaningful contribution from circulating hematopoietic progenitors and renewed by local proliferation, at an estimated median rate of 28% per year by modeling data from ¹⁴C microglial birth dating. It is, however, plausible that PU.1⁺ myeloid cells originating from the bone marrow may enter the brain in particular during pathological processes. Accordingly, peripheral blood hematopoietic cells from non-dementia controls and AD patients were also analyzed to determine the origin (e.g., local or hematopoietic) of potential clones.

### Alzheimer's Disease

Alzheimer's disease (AD) is a complicated, multifactorial, progressive neurodegenerative disorder of the brain, which is characterized by memory deterioration, behavioral disturbances, impairment of activities of daily living, and loss of independent function. AD is the most common form of dementia and although its prevalence is much higher in the older population, it is still the most frequent form of dementia under the age of 65 years. AD can be further categorized based on the age of onset: late onset AD (i.e., onset at >65 years); early onset AD (i.e., onset at ≤50 years); and intermediate onset AD (i.e., onset between 50-65 years).

Symptoms of AD include, but are not limited to, cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders.

Subjects at risk for or predisposed for the development of AD can be identified by, e.g., any one or a combination of diagnostic or prognostic assays known in the art. Although increasing age is the greatest known risk factor for AD, the disease is multifactorial and a number of other risk factors have been identified, including family history. First-degree relatives of patients with AD are more likely to develop the disease. In addition, the presence of risk genes, such as APOE4, may increase the likelihood of developing the disease.

Although a definitive diagnosis of AD can only be made in a post-mortem neuropathologic evaluation (through the detection of extracellular amyloid plaques and intracellular neurofibrillary tangles in brain tissue), clinical Alzheimer's disease in humans may be diagnosed by a combination of symptoms and the results of certain tests to rule out other conditions before making a diagnosis. Medical evaluations typically include patient history, physical examination, and neuropsychological testing. To diagnose a subject as having AD, tests involving memory, problem solving, attention, counting, language, balance, senses, and reflexes may be conducted. Standard medical tests, such as blood and urine tests, or brain scans may be conduced to rule out other possible causes of the symptoms. To confirm a diagnosis of AD, the following must be present and severe enough to affect daily activities: gradual memory loss and progressing cognitive impairment. In some cases, genetic testing may be appropriate. For example, the APOE4 risk allele is associated with higher chances of individuals over the age of 55 years developing AD and could indicate the likelihood of developing the disease. Additional emerging tests may also enable the assessment of biomarkers in people who may be at risk of AD.

### Prophylactic and Therapeutic Methods

The following discussion is presented by way of example only, and is not intended to be limiting.

One aspect of the present technology provides a method for preventing or delaying the onset of Alzheimer's disease or signs or symptoms of AD in a subject predisposed to the development or at risk of having AD (e.g., first-degree relatives of patients with AD characterized by intermediate onset and lacking one or both APOE4 risk alleles).

Subjects at risk for AD can be identified by, e.g., any one or a combination of diagnostic or prognostic assays known in the art. In prophylactic applications, MAPK inhibitors may be administered to a subject susceptible to, or otherwise at risk of AD in an amount sufficient to eliminate or reduce the risk, or delay the onset of the disease, including biochemical and/or behavioral symptoms of the disease, its complications and intermediate pathological phenotypes presenting during development of the disease. Administration of a prophylactic MAPK inhibitor, alone or in combination with additional therapeutic agents, can occur prior to the manifestation of symptoms characteristic of the disease, such that the disease is prevented, or alternatively, delayed in its progression.

Another aspect of the present technology includes methods of treating AD in a subject diagnosed as having, suspected of having, or at risk of having AD. In therapeutic applications, compositions comprising MAPK inhibitors are administered to a subject suspected of, or already suffering from the disease (such as, e.g., subjects exhibiting one or more signs or symptoms associated with AD, and/or lacking one or both APOE4 risk alleles) in an amount sufficient to cure, or at least partially arrest, the symptoms of the disease, including its complications. In some embodiments, the present technology includes methods of providing a tailored treatment approach for a subpopulation of AD subjects. For example, subjects in this subpopulation may be characterized by one or more of intermediate onset AD, a non-carrier of the APOE4 risk alleles or having only one APOE4 risk allele, and exhibiting one or more signs or symptoms of AD, such as cognitive dysfunction.

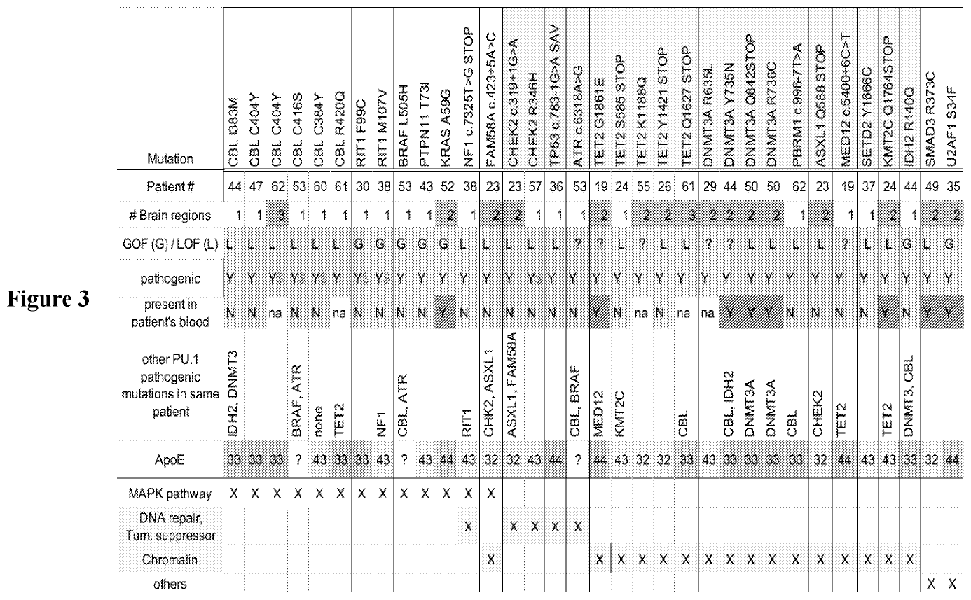

In addition, the disclosure of the present technology contemplates the use of other therapeutic methods known in the art for modulating gene expression (e.g., to increase or decrease the expression of a particular gene or genes). These methods include but are not limited to gene therapy techniques (e.g., the use of viral or non-viral gene delivery systems, RNAi, etc.) to target any one or more of the genes shown to be associated with AD as described herein and as shown in, e.g., FIG. 3. These include genes implicated in the MAPK pathway (CBL, RIT1, BRAF, PTPNJ1, KRAS, NF1, and FAM58A), genes associated with DNA repair or tumor suppression (CHEK2, TP53, and ATR), genes associated with clonal hematopoiesis (FAM58A, TET2, DNMT3A, PBRM1, ASXL1, MED12, SETD2, KMT2C, and IDH2), and others including SMAD3 and U2AFL.

### Combination Therapy

In some embodiments, MAPK inhibitors may be combined with one or more additional therapies related to the treatment of (including without limitation, the inhibition of, prevention of, amelioration of, or delaying the onset of) signs, symptoms, or severity of AD in a subject. In some embodiments, the subject is suspected of having, is at risk for, or is diagnosed as having intermediate onset AD, a non-carrier of one or both of the APOE4 risk alleles, and/or displaying one or more signs or symptoms of AD. Additional therapeutic agents include, but are not limited to, BRAF inhibitors, MEK inhibitors, and/or CSF-1R inhibitors. In some embodiments, the BRAF inhibitor is selected from the group consisting of vemurafenib, dabrafenib, encorafenib, PLX7904, PLX8394, GDC-0879, LGX818, and PLX4720, the MEK inhibitor is selected from the group consisting of AZD8330, refametinib, E6201, MEK162 (binimetinib), PD0325901, pimasertib, R04987655, selumetinib, TAK-733, GDC-0623, WX-544, cobimetinib, and trametinib, and the CSF-1R inhibitor is selected from the group consisting of PLX5622, GW2580, BLZ945, pexidartinib (PLX3397), ARRY-382, PLX7486, and JNJ-40346527.

In some embodiments, when an additional therapeutic agent is administered to a subject in combination with a MAPK inhibitor, a synergistic therapeutic effect is produced. For example, administration of the MAPK inhibitor or mixture of MAPK inhibitors with one or more additional therapeutic agents will have greater than additive effects in the treatment of the disease. For example, lower doses of one or more of any individual therapeutic agent may be used in treating or preventing AD, resulting in increased therapeutic efficacy and decreased side-effects.

### Modes of Administration and Effective Dosages

Any method known to those in the art for contacting a cell, organ, or tissue with a MAPK inhibitor may be employed. Exemplary MAPK inhibitors include, but are not limited to, Gefitinib, Erlotinib, Lapatinib, Sunitinib, Sorafenib, Nilotinib, Dasatinib, Imatinib, Tipifarnib, Sorafenib, U0126, PD184352, AZD6244, BIX02188, BIX02189, SB203580, SB202190, BIRB-796, LY2228820, VX-702, VX-745, and TAK-715. Suitable methods include in vitro, ex vivo, or in vivo methods. In vivo methods typically include the administration of MAPK kinase inhibitors, such as those described above, to a mammal, suitably a human. When used in vivo for therapy, the MAPK inhibitor inhibitors are administered to the subject in effective amounts (i.e., amounts that have desired therapeutic effect). The dose and dosage regimen will depend upon the degree of the disease in the subject, the characteristics of the particular MAPK inhibitor used, e.g., its therapeutic index, the subject, and the subject's history.

The effective amount may be determined during pre-clinical trials and clinical trials by methods familiar to physicians and clinicians. An effective amount of a particular MAPK inhibitor useful in the methods of the present technology may be administered to a mammal in need thereof by any of a number of well-known methods for administering pharmaceutical compounds. The MAPK inhibitor may be administered systemically or locally.

Pharmaceutical compositions are typically formulated to be compatible with its intended route of administration. Examples of routes of administration include, but are not limited to orally, intranasally, parenterally (e.g., intravenously, intramuscularly, intraperitoneally, intradermally, or subcutaneously), systemically, transdermally, iontophoretically, intradermal, intraocularly, ophthalmically, intrathecally, intracerebroventricularly, transmucosally, intravitreally, or topically.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the MAPK inhibitor, sufficient for achieving a therapeutic or prophylactic effect, ranges from about 0.000001 mg per kilogram body weight per day to about 10,000 mg per kilogram body weight per day. In some embodiments, the dosage ranges are from about 0.0001 mg per kilogram body weight per day to about 100 mg per kilogram body weight per day. In some embodiments, the dosage ranges are from about 0.0001 mg per kilogram body weight per day to about 50 mg per kilogram body weight per day. For example dosages can be 1 mg/kg body weight, 10 mg/kg body weight, or 50 mg/kg body weight every day, every two days or every three days or within the range of 1-50 mg/kg every week, every two weeks, or every three weeks. In one embodiment, a single dosage of a MAPK inhibitor ranges from 0.001-10,000 micrograms per kg body weight. In one embodiment, MAPK inhibitor concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regimen.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Examples

The following examples are provided to further illustrate the methods of the present disclosure. These examples are illustrative only and are not intended to limit the scope of the disclosure in any way.

### Materials and Methods

Tissue samples. The study was conducted according to the Declaration of Helsinki, and human tissues were obtained with patient-informed consent and used under approval by the Institutional Review Boards from Memorial Sloan Kettering Cancer Center (IRB protocols #X19-027). Fresh frozen human brain and matched blood were provided by the Netherlands Brain Bank and Human Brain and Spinal Fluid Resource Center [HBSFRC], the Human Brain Collection Core (HBCC, NIH), Hospital Sant Joan de Deu and the Rapid Autopsy Program (MSKCC, IRB #15-021). Samples obtained were clinically and neuropathologically classified by the collaborating institutions as Alzheimer's disease (AD) or non-dementia controls. Samples obtained were clinically and neuropathologically classified as unaffected controls and Alzheimer's disease (AD). Non-dementia patients were grouped into two control groups (below and above 55 years old). The mean age of AD patients was 65 years old and did not present with germline pathogenic PSEN1/2/3 or APP mutations.

Nuclei isolation from frozen brain samples, FACS-sorting and DNA extraction. All samples were handled and processed under Air Clean PCR Workstation. Approximately 250-400 mg of frozen brain tissues were homogenized with a sterile Dounce tissue grinder using a sterile non-ionic surfactant-based buffer to isolate cell nuclei (250 mM Sucrose, 25 mM KCL, 5 mM MgCl2, 10 mM Tris buffer pH 8.0, 0.1% (v/v) Triton X-100, 3 μM DAPI, Nuclease Free Water). Homogenate was filtered in a 40-μm cell strainer and centrifuged 800 g 8 min 4° C. Pellet was gently resuspended in 200 μl of FACS buffer (0.5% BSA, 2 mM EDTA) and incubated on ice for 10 min. After centrifugation 800 g 5 min 4° C., sample was incubated with anti-NeuN (neuronal marker, 1:500, Anti-NeuN-PE, clone A60 Milli-Mark™) for 40 min. After centrifugation 800 g 5 min 4° C., sample was washed with 1× Permeabilization buffer (Foxp3/Transcription Factor Staining Buffer Set, eBioscience™) and centrifuged 1300 g 5 min, without breaks. Staining with anti-Pu.1 antibody in 1× Permeabilization buffer (microglia marker 1:50, Pu.1-AlexaFluor 647, 9G7 Cell Signaling) was performed for 40 min. After a wash with FACS buffer, samples were ready for sorting. Nuclei were FACS-sorted in a BD FACS Aria with a 100-μm nozzle and a sheath pressure 20 psi, operating at ˜1000 events per second. Nuclei were sorted into 1.5 ml certified RNAse, DNAse DNA, ATP and Endotoxins tubes containing 100 l of sterile PBS. For each population >105 nuclei were sorted. Nuclei pellets were centrifuged 20 min at 6000 g and processed immediately for gDNA extraction with QIAamp DNA Micro Kit (Qiagen) following manufacture instructions. DNA from whole-blood samples was extracted using the same protocol. Flow cytometry data was collected using DiVa 8.0.1 Software. Subsequent analysis was performed with FlowJo_10.6.2

Library preparation and sequencing. DNA samples were submitted to the Integrated Genomics Operation (IGO) at MSKCC for quality and quantity analysis, library preparation and sequencing. DNA quality mas measured with Tapestation 2200. All samples had a DNA Integrity Number (DIN) >6. After PicoGreen quantification, ˜200 ng of genomic DNA were used for library construction using the KAPA Hyper Prep Kit (Kapa Biosystems KK8504) with 8 cycles of PCR. After sample barcoding, 2.5 ng-1 μg of each library were pooled and captured by hybridization with baits specific to either the HemePACT (Integrated Mutation Profiling of Actionable Cancer Targets related to Hematological Malignancies) assay, designed to capture all protein-coding exons and select introns of 576 (2.88 Mb) commonly implicated oncogenes, tumor suppressor genes and/or Heme/Brain-PACT (717 genes, 3.44 Mb) an expanded panel that included additional custom targets related to neurological diseases. Capture pools were sequenced on the HiSeq 4000, using the HiSeq 3000/4000 SBS Kit (Illumina) for PE100 reads. Samples were sequenced to a mean depth of coverage of 1106× (Control samples: 1071×, AD samples 1100×).

Mutation data analysis. The data processing pipeline for detecting variants in Illumina HiSeq data is as follows. First the FASTQ files are processed to remove any adapter sequences at the end of the reads using cutadapt (v1.6). The files are then mapped using the BWA mapper (bwa mem v0.7.12). After mapping the SAM files are sorted and read group tags are added using the PICARD tools. After sorting in coordinate order the BAM's are processed with PICARD MarkDuplicates. The marked BAM files are then processed using the GATK toolkit (v 3.2) according the best practices for tumor normal pairs. They are first realigned using ABRA (v 0.92) and then the base quality values are recalibrated with the BaseQRecalibrator. Somatic variants are then called in the processed BAMs using MuTect (v1.1.7) for SNV and ShearwaterML.

MuTect. To identify somatic variants and eliminate germline variants, the pipeline was run as follows: PU.1, DN, and Blood samples against matching-NeuN samples, and NeuN samples against matching-PU.1. In addition, all samples were run against a Frozen-Pool of 10 random genomes. Single Nucleotide Variations (SNVs) mutations were selected [Missense, Nonsense, Splice Site, Splice Regions] that were supported by at least 4 or more mutant reads and with coverage of 50× or more. By screening manually at the fill-out file for each project (˜27 samples per sequencing pool), mutations with low background noise were kept (for which no other unrelated sample sequenced together showed 3 or more counts). This resulted in 342 mutations (missense, nonsense, Splice_site, Splice region).

ShearwaterML, was used to look for low allelic frequency somatic mutations as it has been shown to efficiently call mutations present in a small fraction of cells with true positives being ˜90%. Briefly, the basis of this algorithm is that it uses a collection of deep-sequenced samples to learn for each site a base-specific error model, by fitting a beta-binomial distribution to each site combining the error rates across all normal samples both the mean error rate at the site and the variation across samples, and comparing the observed mutation rate in the sample of interest against this background model using a likelihood-ratio test. In the data set, for each cell type (NeuN, DN, PU.1), “normal” was used as a combination of the other cell types (from Histiocytosis as well as control samples), i.e., PU.1 vs NeuN+DN, DN vs NeuN+PU.1, NEUN vs PU.1⁺ DN, Blood vs NeuN+DN. Since all samples were processed and sequenced the same way, the background error was expected to be even across samples. More than 400 samples were used as background, leading to an average background coverage >400.000×. Resulting variants for each cell type were filtered out as germline if they were present in more than 20% of all reads across samples. Additionally, mutations with coverage of less than 50× and more than 35% variant allelic frequency (VAF) were removed from downstream analysis. p-values were corrected for multiple testing using Benjamini & Hochberg's False Discovery Rate (FDR) and a q-value of cutoff of 0.01 was used to call somatic mutations. Mutations were required to have at least one supporting read in each strand. Somatic mutations within 10 bp of an indel were filtered out as they typically reflect mapping errors. Single Nucleotide Variations (SNVs) mutations [Intronic, Intergenic, Missense, Nonsense, Splice Site, Splice Regions] were selected that were supported by at least 4 or more mutant reads and annotated them using VEP. Finally, mutations with a MAF (minor allelic frequency) cutoff of 0.01 were excluded using the gnomeAD database. This resulted in 446 SNVs.

The final mutant calls of MuTect1 and ShearwaterML were compared and it was found that 30% of the events (96 mutations) that were called by MuTect1 were also called by ShearwaterML. A total of 692 unique variants were found, with a mean coverage at the mutant site of 656× (10% percentile: 277×, 90% percentile: 1167×) and a mean of 22 mutant reads (10% percentile: 4, 90% percentile: 36), and 81.8% of mutated supported by at least 5 mutant reads.

Validation of mutations by digitalPCR. Validation of 10.2% of mutations (71/692) was performed by droplet-digital PCR (ddPCR) on pre-amplified DNA or on libraries (when DNA not available) and confirmed 63/71 of mutations tested (˜90%). Most assays were performed in all cell types isolated from the same brain region and matching blood. The mean depth of ddPCR was ˜4000×, mutant counts of 3 or more were considered positive. VAF obtained by ddPCR correlated with original VAF by sequencing (R2 0.93, p<0.0001; see FIG. 5C). For KRAS_G12D: Bio-Rad validated assay (Unique Assay ID: dHsaMDV2510596) and MTOR_Arg1616His_c.4847G>A: Bio-Rad validated assay (Unique Assay ID: dHsaMDV2510596). Other assays specific for the detection of these mutations were designed and ordered through Bio-Rad. For newly designed assays, cycling conditions were tested to ensure optimal annealing/extension temperature as well as optimal separation of positive from empty droplets. All reactions were performed on a QX200 ddPCR system (Bio-Rad catalog #1864001). When possible, each sample was evaluated in technical duplicates or quartets. Reactions contained 10 ng gDNA, primers and probes, and digital PCR Supermix for probes (no dUTP). Reactions were partitioned into a median of ˜31,000 droplets per well using the QX200 droplet generator. Emulsified PCRs were run on a 96-well thermal cycler using cycling conditions identified during the optimization step (95° C. 10′; 40-50 cycles of 94° C. 30′ and 52-56° C. 1′; 98° C. 10′; 4° C. hold). Plates were read and analyzed with the QuantaSoft software to assess the number of droplets positive for mutant DNA, wild-type DNA, both, or neither.

Quantification of mutation burden quantification and classification. Overall, a total of 692 mutations were detected resulting in 0.33 mutations/Mb sequenced, compatible with the mutation burden estimated in normal tissues and low grade tumors. To quantify mutation burden, the inventors took into consideration that the same samples were sequenced with Heme-PACT (2.88 Mb) or the extended panel Heme/Brain-PACT (3.44 Mb) and normalized the number of mutations per sample per the Mb sequenced for that specific sample (or the sum of Mb sequenced in each cell type, to make the calculation of mutations per Mb per Patient in the different cell types).

Classification of variants. To classify variants according to their pathogenicity the following was performed: variants were classified as “predicted pathogenic” when classified as such by 4 pathogenicity predictions [PolyPhen-2, SIFT, CADD-MSC (high), FATHMM (Functional Analysis through Hidden Markov Models (v2.3)]. Variants were classified as “pathogenic” if shown by ClinVar and/or OncoKb. These two databases report pathogenicity based on supporting evidence from curated literature. Classical MAPK-pathway genes are considered to be those reported to be mutated in RASopathies.

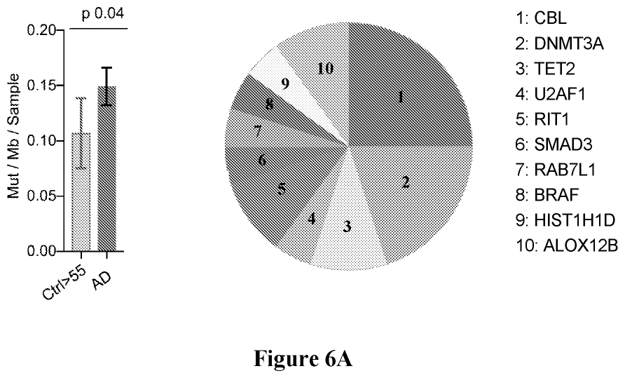

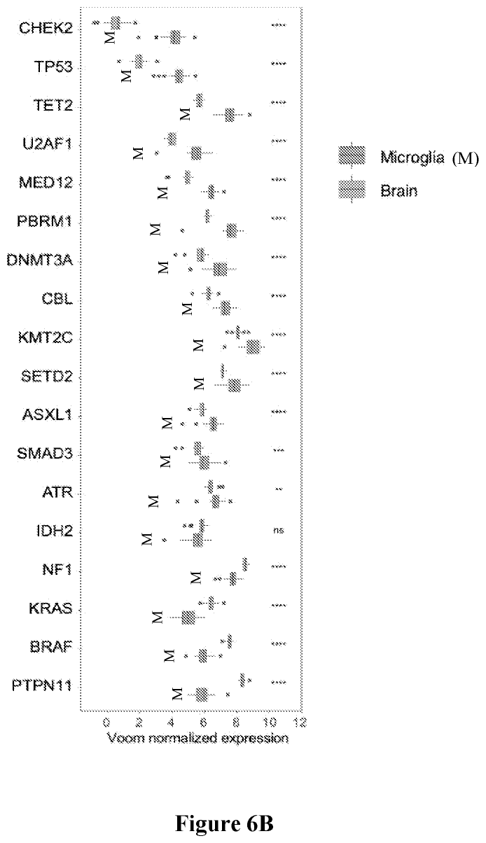

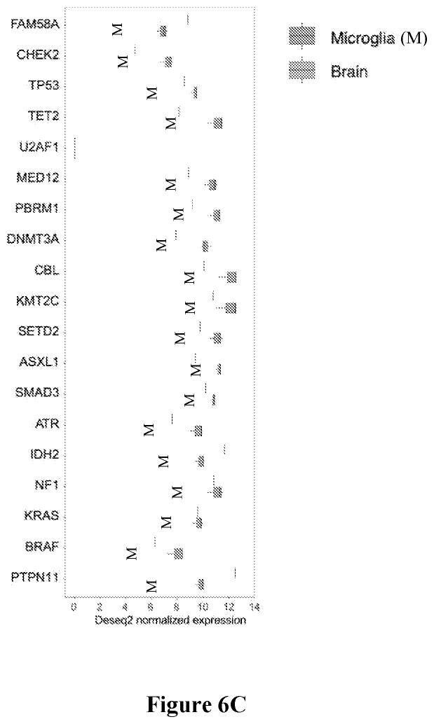

RNA-seq datasets. The following publicly available datasets were used in this study to investigate the gene expression levels of gene targets of somatic mutations found in AD patients as well as heathy individuals (FIGS. 6B-6C): (1) Galatro et al., (GSE99074), normalized gene expression data and associated clinical information of isolated human microglia (N=39) and whole brain (N=16) from healthy controls were downloaded from GEO; (2) Gosselin et al., raw gene expression data and associated clinical information of isolated microglia (N=3) and whole brain (N=1) from healthy controls were extracted from the original dataset. Raw counts were normalized using DESeq2 package in R.

Immunofluorescence in human AD samples. Paraffin-fixed brain sections from 10 AD patients and 10 age-matched controls (a gracious gift from Dr Marco Prinz, Freiburg) and paraffin-fixed brain sections from brain lesions from Erdheim-Chester Disease patients (ECD were stained with anti-pERK1/2 (1:200, sc-136521, Santa Cruz) or anti-CD163 (0.06 μg ml-1, 760-4437, Cell Marque), rabbit anti-pERK1/2 (1 μg ml-1, 4370, Cell Signaling). Secondary Alexa-were added at 1:200. Images were taken with BZ-9000 BIOREVO microscope (Keyence) and analyzed using the BZ-II Analyzer (Keyence) or with a LSM880 Zeiss microscope with 40×/1.4 (oil), performing a tile scan and z stack on the whole tissue at a 512×512 or 1,024×1,024 pixel resolution and manually analyzed using Imaris (Bitplane) software.

Statistical analysis. Statistical significance was analyzed with GraphPad Prism (v8) using unpaired two-tailed Mann-Whitney U test and one-way ANOVA and Tukey test for multiple comparisons, two-tailed Fisher Exact test and Pearson's. Significance was considered at P<0.05.

Data availability. Raw sequencing data (BAM files) are deposited in the NCBI database SRA (accession number pending).

### Biochemical Analysis

Cell culture: HEK 293T cells (ATCC) were maintained in Dulbecco's modified Eagle's medium (Mediatech, Inc.) supplemented with 10% fetal bovine serum (Sigma) and 1000 IU/ml penicillin, 1000 IU/ml streptomycin.

Cloning and Site-directed mutagenesis: The expression vectors for Flag-tagged CHK2 kinase and RIT1 were from Sino Biological and Origene, respectively. The vector encoding HA-tagged c-Cbl was a kind gift from Dr. Nicholas Carpino (Stony Brook). Site-directed mutagenesis was performed using the QuikChange Kit (Agilent).

Cell transfection, Immunoprecipitation, and Western Blotting: HEK293T cells were transfected 24 hours after plating with 2.5 μL of Mirus Transit LT1 per μg of DNA. Cells were harvested and lysed 48 hrs after transfection using a buffer containing 25 mM Tris, pH 7.5, 1 mM EDTA, 100 mM NaCl, 1% NP-40, 10 μg/ml leupeptin, 10 μg/ml aprotinin, 200 μM PMSF, and 0.2 mM Na₃VO₄. For EGF stimulation, the media was replaced 24 hours after transfection with DMEM containing 1% FBS and antibiotics. After a further 24 hours in this starvation media, the cells were stimulated with 50 ng/ml EGF for 5 minutes at 37° C. For Western blotting, lysates were resolved by SDS-PAGE, transferred to PVDF membranes, and probed with the appropriate antibodies. Horseradish peroxidase-conjugated secondary antibodies (GE Healthcare) and Western blotting substrate (Thermo) were used for detection. For anti-Cdc42 immunoprecipitation experiments, cell lysates (1 mg total protein) were incubated overnight with 1 μg of anti-Cdc42 antibody (Santa Cruz) and 25 μL of protein A agarose (Roche) at 4° C. Anti-Flag immunoprecipitations were done with anti-Flag M2 affinity resin (Sigma). The beads were washed three times with lysis buffer, then eluted with SDS-PAGE buffer and resolved by SDS-PAGE. The proteins were transferred to PVDF membrane for Western blot analysis.

Immunoprecipitation Kinase assay: Cell lysates (1 mg protein) were incubated overnight with 30 μL of anti-Flag M2 affinity resin on a rotator at 4° C., then washed three times with Tris-buffered saline (TBS). A portion of each sample was eluted with SDS-PAGE sample buffer and analyzed by anti-Flag Western blotting. The remaining sample was used for a radioactive kinase assay. The immunoprecipitated proteins were incubated with 25 μL of reaction buffer (30 mM Tris, pH 7.5, 20 mM MgCl2, 1 mg/mL BSA, 400 μM ATP), 650 μM CHKtide peptide (KKKVRSGLYRSPSMPENLNRPR (SEQ ID NO: 1), SignalChem), and 50-100 cpm/pmol of [γ³²-P] ATP at 30° C. for 15 minutes. The reactions were quenched using 45 μL of 10% trichloroacetic acid. The samples were centrifuged and 30 μL of the reaction was spotted onto Whatman P81 cellulose phosphate paper. After washing with 0.5% phosphoric acid, incorporation of radioactive phosphate into the peptide was measured by scintillation counting.

PAK1 CRIB binding assay: The Cdc42/Rac interactive binding (CRIB) domain from PAK1 was expressed as a GST fusion protein in E. coli and purified with glutathione-agarose. The immobilized CRIB domain was incubated overnight with cell lysates (0.5 mg total protein) at 4° C. The resin was washed three times with lysis buffer, and bound proteins were eluted with SDS-PAGE sample buffer and analyzed by anti-RIT1 Western blotting.

### Example 1: Microglia Carry a Greater Clonal Diversity than Neurons and Glia

This experiment analyzed 665 samples from brain cell types isolated mainly from the hippocampus, cortex, brainstem, and cerebellum of 80 individuals, and matching blood when available, including 45 patients (323 samples) with neuropathologically proven sporadic AD (mean age 65), and 35 individuals without dementia (aged <55 n=16, 196 samples, and age>55 n=19, 146 samples, FIG. 5A). Nuclei corresponding to brain cell types, PU.1⁺ (myeloid/microglia, ˜5%), NeuN+(neurons, ˜45%), PU.1⁻/NeuN (double negative, DN, glial cells, ˜40%) (FIG. 1A, and FIG. 5B) were separated and collected by cell sorting (˜105 nuclei/sample). DNA extracted from these samples and matching blood was submitted to targeted hybridization/capture and deep-DNA sequencing at a mean coverage of ˜1100× for a panel of 716 genes implicated in tumoral malignancies{Cheng, 2015 #141}{Durham, 2019 #35} and neurological diseases (HEME/BRAIN-PACT, 3.43 Mb, FIG. 1B, see Methods). A total of 692 mutations (0.33 mut/Mb) were detected in 665 samples with a mean allelic frequency (AF) of 3.25% (median 1.35%) (FIG. 1B, see Methods). To test the reproducibility of the sequencing and analysis approach, ddPCR on pre-amplified DNA was performed, which confirmed mutations in 63/71 cases (90%) (FIG. 1B and FIG. 5C, see Methods). Overall, brain mutations at AF>0.3% are ˜four times more frequent in PU.1⁺ cells (˜0.5/Mb) than in NeuN⁺ (˜0.13/Mb) neurons and DN cells (˜0.15/Mb) (p<0.0001, FIG. 1C), irrespective of age or disease (FIG. 1D). As expected, the frequency of blood mutations increases with age (˜0.6/Mb<55 & 1.2/Mb >55, FIG. 1D). Most (97.5%) mutations are cell type specific (FIG. 1E), which was confirmed by ddPCR in 10% of mutations and few mutations are shared between the blood and brain PU.1⁺ cells from older individuals, corresponding to clonal hematopoiesis (CH) (FIG. 1E, and see below). These data indicate that somatic clones are more frequent among brain microglia than in neurons and other glia, and overall is consistent with the hypothesis that brain microglia proliferate locally with little input from circulating hematopoietic precursors in younger individuals, while contamination or engraftment of circulating cells is observed in a fraction of older patients.

### Example 2: Somatic Clones Carrying Pathogenic Driver-Mutations are Found in Large Excess in the Microglia from AD Patients with Low APOE4 Genetic Risk

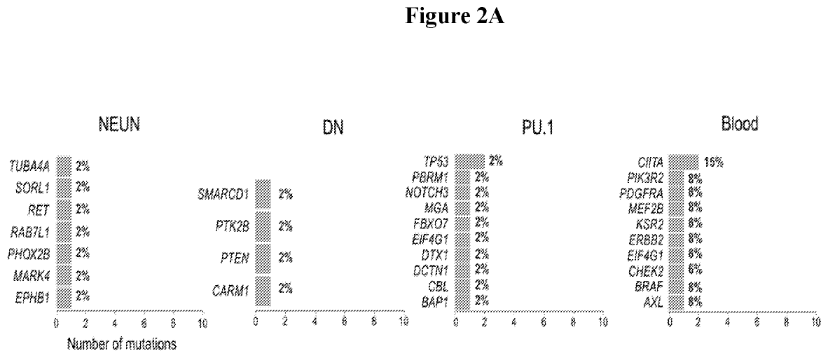

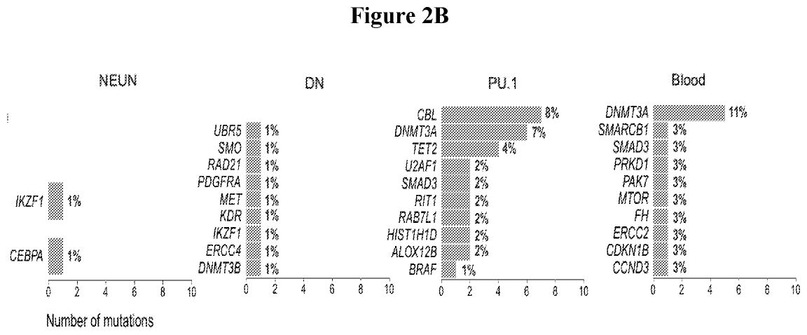

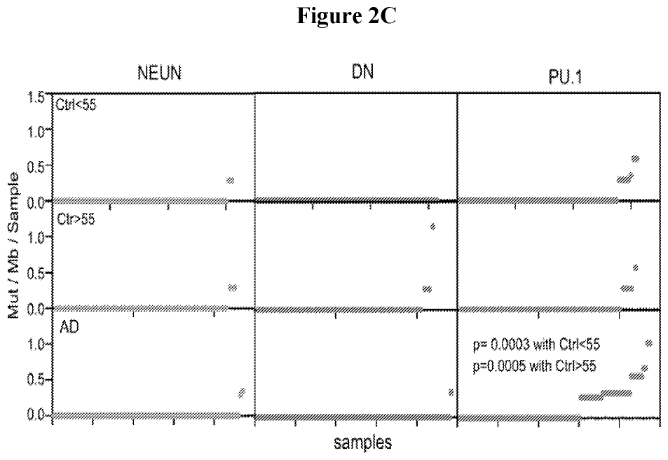

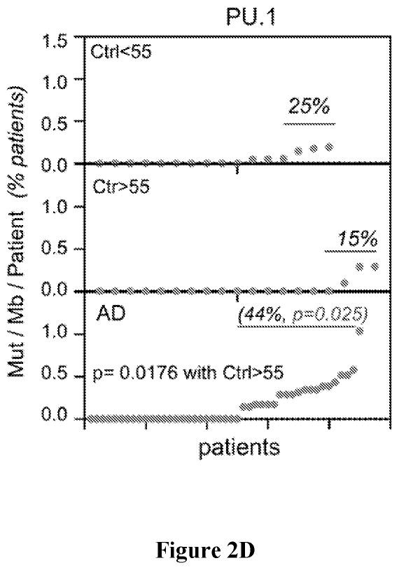

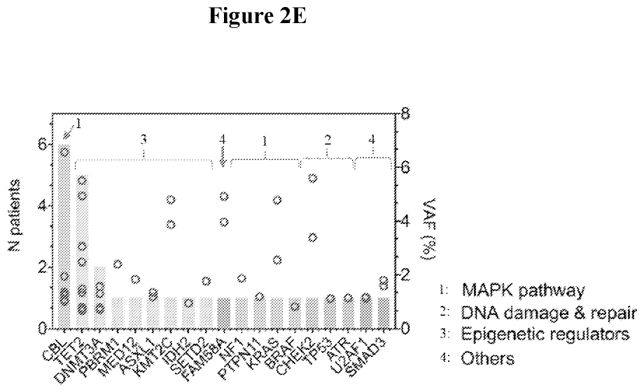

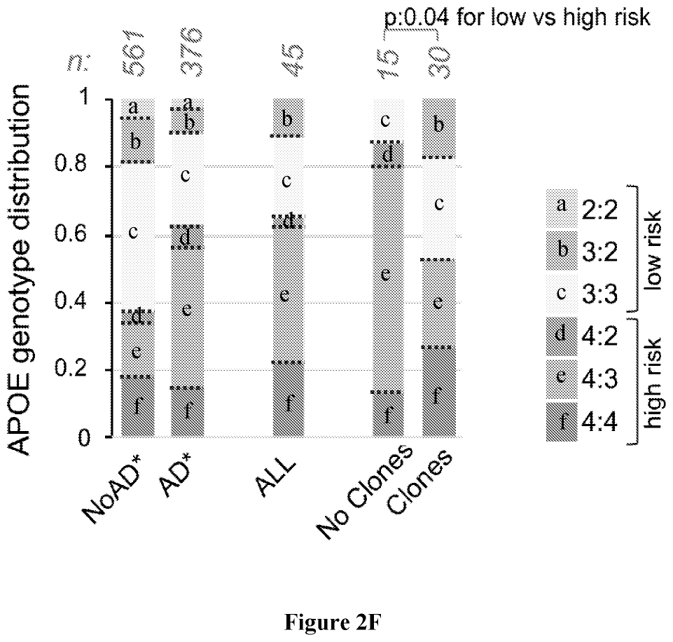

There was no difference in the overall number of mutations detected in the brain of controls and AD patients (FIG. 1D), but PU.1⁺ samples from AD accumulated more mutations predicted to be potentially deleterious for gene function by 4 modeling predictors (Polyphen, SIFT, CADD/MSC and COSMIC) per Mb in comparison to age-matched controls (p=0.04 FIG. 6A), with the gene CBL being the most recurrent target (FIGS. 2A-2B). This analysis did not identify recurrent mutations in neurons and glia (FIGS. 2A-2B). More significant, independent analysis of mutations reported to be pathogenic for gene function and associated with human diseases by ClinVar and OncoKB (herein reported as “pathogenic” or “driver” mutations) shows a large excess of these pathogenic mutations per Mb in PU.1⁺ samples from AD patients in comparison to age-matched controls (p<0.0005), but not in NeuN and DN samples (FIG. 2C). Moreover, a higher proportion of AD patients carry PU.1⁺ clones with pathogenic mutations in comparison to age-matched controls (p=0.0176) (FIG. 2D). Overall, PU.1⁺ clones carrying pathogenic mutations were detected in 20 out of 45 patients (44%, p=0.025 in comparison to age-matched controls) (FIG. 2D), at allelic frequencies between 1 and 5% (likely corresponding to 2 to 10% of microglia), with CBL being the most mutated gene again (FIG. 2E). Genes targeted by these driver mutations are all expressed in the brain and the majority are enriched in microglia (FIGS. 6B-6C). In addition, while the APOE genotype of the cohort of AD patients is comparable to published allelic frequencies with 62% of patients carrying the APOE4 allele, the frequency of APOE4 carriers is only ˜50% among patients with somatic clones carrying driver mutations, versus 85% of patients without predicted/pathogenic clones (p:0.04, FIG. 2F). Taken together, these data show that brains of AD patients harbor a large excess of microglia PU.1⁺ clones carrying pathogenic mutations, as compared to age-matched controls, while, interestingly, patients carrying these microglial clones are less likely to carry the apolipoprotein ε4 risk allele (APOE4), which is compatible with a possible role of mutant microglia in the pathogenesis of AD in these patients.

Driver mutations (47 in 20 patients) can be divided into 3 main categories (FIG. 2E). Twenty-six mutations in 11 patients comprise recurrent TET2 mutations (in 5 patients) and DNMT3A mutations (in 4 patients) as well as KMT2C, SETD2, IDH2, ASXL1, MED12 and PBRM1 mutations (one patient each) (FIGS. 2E and 3). In addition, one patient was found to have an oncogenic SMAD3 loss-of-function mutation (FIGS. 2E and 3). These genes are associated with clonal hematopoiesis (CH) and mutations were frequently detected in the blood of the same patients (FIG. 3). CH is associated with an increased risk of myeloid leukemia and has also been epidemiologically linked to increased cardiovascular risk. However, there is no available data linking CH to dementia or to AD, and at present it cannot be excluded that the presence of blood clones in the brains of AD patients could represent leucocyte recruitment to a diseased brain.

### Example 3: Pathogenic Microglial Clones Carry Mutations that Activate the MAPK Pathway

A second category of mutations involve the MAP kinase (MAPK) pathway. As noted above, CBL mutations are the most frequent mutation in this series of patients, with recurrent loss-of-function mutations of CBL in six patients (FIG. 3). CBL is an E3 ubiquitin-protein ligase that negatively regulates growth factor signaling via the MAPK pathway. All CBL mutations (p.R420Q, p.C416S, p.C404Y p.C384Y and p.I383M) occur in the RING-type zinc finger domain (FIG. 4A). CBL R420Q was previously identified in acute myeloid leukemia and RASopathies and causes a loss of CBL function leading to increased growth factor signaling, similar to CBL Y371H. The CBL I383M, CBL C404Y, and CBL C416S mutations have been associated with myeloproliferative disorders and RASopathies, but have not been functionally characterized and the C384Y mutation is novel, but a related (C384R) mutant is found in juvenile myelomonocytic leukemia and causes loss of CBL function. Therefore, HA-tagged WT and mutant forms of CBL were expressed in HEK293T cells. After stimulation with EGF, the classical Y371H mutant as well as C404Y, C416S, and (to a lesser extent) C384Y mutants showed increased MAP kinase phosphorylation relative to WT CBL (FIGS. 3 and 4B). Therefore, the CBL loss-of-function mutations identified here as being enriched in AD patients activate the MAP kinase pathway. In addition, CBL mutations are not detected in the patients' blood, suggesting they are private to microglia. A PU.1⁺ CBL pathogenic clone was also found in a brain from the cohort of age-matched control, albeit it is of note that this individual had neuropathological lesions of AD (Braakl).

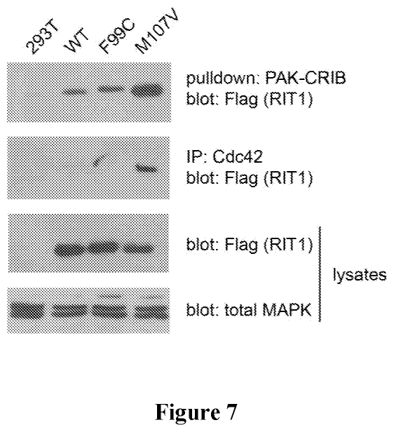

In addition to CBL, PU.1⁺ cells from AD patients harbor 4 deleterious mutations in other genes that belong to the classical MAPK pathway and are found mutated in RASopathies (and ClinVar) (FIGS. 2E and 3). Gain-of-function mutations of BRAF, RAS, PTPN11, and loss-of-function mutations of NF1, which are positive and negative regulators of the MAPK pathway, respectively, were found in one patient each. Mutations in these genes have been previously associated with myeloid malignancies, RASopathies, and cognitive dysfunction. Moreover, 2 patients presented with mutations in RIT1 (F99C and M107V), predicted to be pathogenic, but not previously described. RIT1 mutations were not found among controls. Germline activating mutations in RIT1 lead to enhanced MAP kinase signaling in RASopathies. Both RIT1 mutations are located in or near the RIT1 Switch II domain (FIG. 4C), a region that undergoes conformational changes in response to GDP/GTP exchange. Flag-tagged wild-type, F99C, and M107V forms of RIT1 were expressed in HEK293T cells, and serum stimulation led to strong increase in MAP kinase phosphorylation in cells expressing the microglia-associated mutants. MAP kinase activation was strongest for the M107V mutant, which showed significant MAP kinase activation in the absence of serum suggesting that it could act independently of growth factor stimulation (FIG. 4C). In immunoprecipitation experiments it was found that the F99C and M107V mutant forms of RIT1 increased binding to PAK1 CRIB domain and to Cdc42 (FIG. 7). These data indicate that loss-of-function mutations in CBL and NF1 as well as activating mutations in RIT1, BRAF, KRAS and PTPN11, respectively, negative and positive regulators of the MAPK pathway (FIG. 4D) are enriched in AD samples in comparison to age-matched controls (p=0.03, FIG. 4E), and carry the potential to activate microglial MAP kinase signaling in 10 patients (22% of patients in the series analyzed). Also, in an additional patient, a pathogenic mutation in FAM58A was identified, which is not a classical MAPK gene, but has been reported to activate the pathway by regulating RAF (FIGS. 3 and 4D).

### Example 4: Microglia from AD Patients Display In Situ ERK Phosphorylation

In contrast to CH mutations, 12/13 of these MAPK activating mutations were not detected in the patient's blood, suggesting they are private to microglia (FIG. 3). These genes are all highly or predominantly expressed in microglia in the brain (FIGS. 6B-6C), and the mutations are detectable in the patient's hippocampus. MAPK activating mutations are present at allelic frequencies ˜1-5%, corresponding to clones of 2 to 10% of mutant microglia, which is comparable to that of the BRAF^{V600E} mutation in the brain of histiocytosis patients with BRA^{V600E}-associated neurodegeneration. Activation of the MAPK pathway has already been proposed to be an early event in the pathophysiology of AD in humans. Thus, to examine if MAP kinase activation is a feature of microglia in these patients, immunostaining for pERK and CD163, which label microglia, was performed in a separate series of tissues from AD patients and controls, and it was observed that pERK is expressed by macrophages from 9/10 AD lesions, as well as histiocytosis patients, but not in microglia from controls (FIG. 4F).

In summary, the data from Examples 1-4 suggest that microglial clones carrying MAPK activating mutations are enriched in brain from patients with AD and may be relevant to the neurodegenerative process. The data also suggests MAPK inhibitors may be useful in methods for treating or preventing AD in subjects suspected of having, at risk for, or diagnosed with AD.

### Example 5: Somatic Loss-of-Function Tumor Suppressor Mutations

A third, smaller, group of pathogenic mutations include DNA repair genes with known tumor suppressor function (FIG. 3). Three patients showed loss-of-function mutations in CHEK2, TP53, and ATR, with the latter associated with a CBL mutation in the same patient. The transcription factor TP53 and the serine/threonine kinase and DNA damage sensors ATR and CHEK2 are mutated in cancer, and germline deficiency causes neurodegeneration. Patient AD23 had a CHEK2 mutation in the splice site donor for intron 2, which would lead to a truncated form of CHK2 lacking the entire protein kinase domain (FIGS. 3 and 4G). Interestingly, another patient (AD57) had a novel R346H mutation within the catalytic loop of the protein kinase domain (FIGS. 3 and 4G). Flag-tagged wild-type and R346H CHK2 were expressed in HEK293T cells and the proteins were isolated by immunoaffinity capture using anti-Flag resin. CHK2 activity was measured with [³²P]-labeled ATP and a synthetic CHK2 substrate peptide. Wild-type CHK2 showed robust activity, while the R346H mutant was inactive (FIG. 4G). This finding was confirmed by Western blotting using an antibody that recognizes the autophosphorylated and activated form of CHK2. In contrast to wild-type CHK2, there was no signal for T383 phosphorylation of the mutant (FIG. 4G), indicating that CHK2 R346H is a loss of function mutant. Therefore, a small fraction of AD patients' PU.1⁺ clones carry somatic loss-of-function tumor suppressor mutations, which germline deficiency has been linked to neurodegeneration, including 2 patients with CHEK2 loss-of-function alleles.

Taken together, the data presented herein suggest the novel concept that a clonal microglial disease could contribute to Alzheimer's disease onset or progression, which raises the possibility that a molecular diagnosis of Alzheimer's disease and the use of targeted therapy, in particular, inhibitors of the MAPK pathway, may contribute to a more personalized and efficient management of a subset or subsets of patients with Alzheimer's disease.

### Example 6: Methods for the Prevention and Treatment of Alzheimer's Disease

This example prophetically demonstrates the use of MAP kinase (MAPK) inhibitors for the prevention and treatment of Alzheimer's disease. One of skill in the art will understand that the example set forth below is illustrative of Alzheimer's disease generally, with methods generally applicable to Alzheimer's disease.

### Animal Models

Animal models suitable for this example include any accepted Alzheimer's disease non-human animal model, including, but not limited to, transgenic and non-transgenic animal models, such as those disclosed in Drummond & Wisniewski (Acta Neuropathol. 133(2):155-175 (2017) and Puzzo, et al. (Biochem Pharmacol 88(4):450-467 (2014)). One of skill in the art will understand that the following description is illustrative and may be applied as appropriate to other animal models.

General. Animal models are selected and maintained according to relevant standards known in the art. Subjects are administered compounds of the present technology (e.g., one or more MAPK inhibitors alone or in combination with one or more active agents described herein) according to methods described herein, such as by oral, i.p., or any other acceptable route of administration. In some embodiments, the compound is administered once daily, once weekly, or once monthly. In some embodiments, compounds are administered multiple times daily, multiple times weekly, or multiple times monthly. Control subjects are administered vehicle alone.

For methods of prevention, subjects are administered compounds of the present technology prior to or subsequent to the development of symptoms and/or pathologies of Alzheimer's disease or related disorders and assessed for reversal of symptoms/pathologies or attenuation of expected symptoms/pathologies using methods known in the art.

Subjects will be monitored for the onset or time to development of one or more signs or symptoms of Alzheimer's disease, survival time, and/or the attenuation of cognitive decline. Acceptable behavioral assays are available for assessing cognitive impairment in animals, such as rodents, and are known in the art. These assays include, but are not limited to, fear conditioning using a fear memory apparatus, the radial arm water maze (RAWM), and the Morris Water Maze (MWM) (see, e.g., Puzzo, 2014).

Subjects will also be assessed for the presence of one or more of the MAPK-activating mutations described herein (e.g., a mutation in one or more of CBL, RIT1, BRAF, PTPN11, KRAS, NF1, and FAM58A, as described herein). To determine whether the subject is characterized by one or more of the MAPK-activating mutations, tissue samples (e.g., blood or brain tissue) will be isolated from the subject and analyzed for the presence of the mutations using any acceptable methods known in the art. Subjects may also be assessed for the presence of APOE4 risk alleles. Subjects may be selected for treatment if they are characterized by one or more MAPK-activating mutations and/or if they are lacking one or both APOE4 risk alleles.

Results: It is expected that MAPK inhibitors will induce reversal of symptoms and/or pathologies of Alzheimer's disease in animal models. These results will show that MAPK inhibitors are useful and effective for the prevention and treatment of Alzheimer's disease. These results will show that MAPK inhibitors are useful and effective for the prevention and treatment of Alzheimer's disease in subjects characterized by one or more of the MAPK-activating mutations described herein.

### Human Subjects

Human subjects diagnosed as having or suspected to have Alzheimer's disease and presently displaying one or more symptoms and/or pathologies of Alzheimer's disease, are recruited using selection criteria known and accepted in the art.

Methods of Prevention and Treatment: Subjects are administered one or more MAPK inhibitors at a dosage and frequency commensurate with the stage and severity of disease. In some embodiments the one or more MAPK inhibitors are administered once daily, once weekly, or once monthly. In some embodiments, the one or more MAPK inhibitors are administered multiple times daily, weekly, or monthly.

For methods of prevention, subjects are administered one or more MAPK inhibitors prior to or subsequent to the development of symptoms and/or pathologies of Alzheimer's disease and assessed for reversal of symptoms/pathologies or attenuation of expected symptoms/pathologies using methods known in the art.

Subjects will also be assessed for the presence of one or more of the MAPK-activating mutations described herein (e.g., a mutation in one or more of CBL, RIT1, BRAF, PTPN11, KRAS, NF1, and FAM58A, as described herein). To determine whether the subject is characterized by one or more of the MAPK-activating mutations, tissue samples (e.g., blood) will be isolated from the subject and analyzed for the presence of the mutations using any acceptable methods known in the art. Subjects may also be assessed for the presence of APOE4 risk alleles. Subjects may be selected for treatment if they are characterized by one or more MAPK-activating mutations and/or if they are lacking one or both APOE4 risk alleles. Subjects selected for treatment with a MAPK inhibitor may also include those who are predisposed to the development or at risk of having AD (e.g., first-degree relatives of patients with AD characterized by intermediate onset and lacking one or both APOE4 risk alleles).

Results: It is expected that the administration of one or more MAPK inhibitors will induce reversal of symptoms and/or pathologies of Alzheimer's disease in human subjects. These results will show that one or more MAPK inhibitors are useful and effective for the prevention and treatment of Alzheimer's disease. These results will show that MAPK inhibitors are useful and effective for the prevention and treatment of Alzheimer's disease in subjects characterized by one or more of the MAPK-activating mutations described herein.

### REFERENCES

1 McQuade, A. & Blurton-Jones, M. Microglia in Alzheimer's Disease: Exploring How Genetics and Phenotype Influence Risk. J Mol Biol 431, 1805-1817, doi:10.1016/j.jmb.2019.01.045 (2019).
2 Nott, A. et al. Brain cell type-specific enhancer-promoter interactome maps and disease-risk association. Science 366, 1134-1139, doi:10.1126/science.aay0793 (2019).
3 Mass, E. et al. A somatic mutation in erythro-myeloid progenitors causes neurodegenerative disease. Nature 549, 389-393, doi:10.1038/nature23672 (2017).
4 Stratton, M. R., Campbell, P. J. & Futreal, P. A. The cancer genome. Nature 458, 719-724, doi:10.1038/nature07943 (2009).
5 Strittmatter, W. J. et al. Apolipoprotein E: high-avidity binding to beta-amyloid and increased frequency of type 4 allele in late-onset familial Alzheimer disease. Proc Natl Acad Sci USA 90, 1977-1981, doi:10.1073/pnas.90.5.1977 (1993).
6 Saunders, A. M. et al. Association of apolipoprotein E allele epsilon 4 with late-onset familial and sporadic Alzheimer's disease. Neurology 43, 1467-1472, doi:10.1212/wnl.43.8.1467 (1993).
7 Fernandes, M. S. et al. Novel oncogenic mutations of CBL in human acute myeloid leukemia that activate growth and survival pathways depend on increased metabolism. J Biol Chem 285, 32596-32605, doi:10.1074/jbc.M110.106161 (2010).
8 Sargin, B. et al. Flt3-dependent transformation by inactivating c-Cbl mutations in AML. Blood 110, 1004-1012, doi:10.1182/blood-2007-01-066076 (2007).
9 Loh, M. L. et al. Mutations in CBL occur frequently in juvenile myelomonocytic leukemia. Blood 114, 1859-1863, doi:10.1182/blood-2009-01-198416 (2009).
10 Hebert, L. E., Weuve, J., Scherr, P. A. & Evans, D. A. Alzheimer disease in the United States (2010-2050) estimated using the 2010 census. Neurology 80, 1778-1783, doi:10.1212/WNL.0b013e31828726f5 (2013).
11 Association, A. s. 2019 Alzheimer's Disease Facts and Figures. https: www.alz.org media documents alzheimers-facts-and-figures-2019-r.pdf (2019).
12 Cruts, M. et al. Molecular genetic analysis of familial early-onset Alzheimer's disease linked to chromosome 14q24.3. Hum Mol Genet 4, 2363-2371, doi:10.1093/hmg/4.12.2363 (1995).
13 Rogaev, E. I. et al. Familial Alzheimer's disease in kindreds with missense mutations in a gene on chromosome 1 related to the Alzheimer's disease type 3 gene. Nature 376, 775-778, doi:10.1038/376775a0 (1995).
14 Levy-Lahad, E. et al. Candidate gene for the chromosome 1 familial Alzheimer's disease locus. Science 269, 973-977, doi:10.1126/science.7638622 (1995).
15 Levy-Lahad, E. et al. A familial Alzheimer's disease locus on chromosome 1. Science 269, 970-973, doi:10.1126/science.7638621 (1995).
16 Chartier-Harlin, M. C. et al. Early-onset Alzheimer's disease caused by mutations at codon 717 of the beta-amyloid precursor protein gene. Nature 353, 844-846, doi:10.1038/353844a0 (1991).
17 Goate, A. et al. Segregation of a missense mutation in the amyloid precursor protein gene with familial Alzheimer's disease. Nature 349, 704-706, doi:10.1038/349704a0 (1991).
18 Lanoiselee, H. M. et al. APP, PSEN1, and PSEN2 mutations in early-onset Alzheimer disease: A genetic screening study of familial and sporadic cases. PLoS Med 14, e1002270, doi:10.1371/journal.pmed.1002270 (2017).
19 Jonsson, T. et al. Variant of TREM2 associated with the risk of Alzheimer's disease. N Engl J Med 368, 107-116, doi:10.1056/NEJMoa1211103 (2013).
20 Guerreiro, R. et al. TREM2 variants in Alzheimer's disease. N Engl J Med 368, 117-127, doi:10.1056/NEJMoa1211851 (2013).
21 Martincorena, I. et al. Tumor evolution. High burden and pervasive positive selection of somatic mutations in normal human skin. Science 348, 880-886, doi:10.1126/science.aaa6806 (2015).
22 Martincorena, I. & Campbell, P. J. Somatic mutation in cancer and normal cells. Science 349, 1483-1489, doi:10.1126/science.aab4082 (2015).
23 Behjati, S. et al. Genome sequencing of normal cells reveals developmental lineages and mutational processes. Nature 513, 422-425, doi:10.1038/nature13448 (2014).
24 Frank, S. A. Evolution in health and medicine Sackler colloquium: Somatic evolutionary genomics: mutations during development cause highly variable genetic mosaicism with risk of cancer and neurodegeneration. Proc Natl Acad Sci USA 107 Suppl 1, 1725-1730, doi:10.1073/pnas.0909343106 (2010).
Poduri, A., Evrony, G. D., Cai, X. & Walsh, C. A. Somatic mutation, genomic variation, and neurological disease. Science 341, 1237758, doi:10.1126/science.1237758 (2013).
26 McConnell, M. J. et al. Intersection of diverse neuronal genomes and neuropsychiatric disease: The Brain Somatic Mosaicism Network. Science 356, doi:10.1126/science.aa11641 (2017).
27 D'Gama, A. M. et al. Somatic Mutations Activating the mTOR Pathway in Dorsal Telencephalic Progenitors Cause a Continuum of Cortical Dysplasias. Cell Rep 21, 3754-3766, doi:10.1016/j.celrep.2017.11.106 (2017).
28 Poduri, A. et al. Somatic activation of AKT3 causes hemispheric developmental brain malformations. Neuron 74, 41-48, doi:10.1016/j.neuron.2012.03.010 (2012).
29 Wei, W. et al. Frequency and signature of somatic variants in 1461 human brain exomes. Genet Med 21, 904-912, doi:10.1038/s41436-018-0274-3 (2019).
30 Park, J. S. et al. Brain somatic mutations observed in Alzheimer's disease associated with aging and dysregulation of tau phosphorylation. Nat Commun 10, 3090, doi:10.1038/s41467-019-11000-7 (2019).
31 Reu, P. et al. The Lifespan and Turnover of Microglia in the Human Brain. Cell Rep 20, 779-784, doi:10.1016/j.celrep.2017.07.004 (2017).
32 Vostrikov, V. M. [Electron-cytochemical study of microglia in Alzheimer's disease and senile dementia]. Zh Nevropatol Psikhiatr Im S S Korsakova 85, 974-976 (1985).
33 Evans, P. H., Yano, E., Klinowski, J. & Peterhans, E. Oxidative damage in Alzheimer's dementia, and the potential etiopathogenic role of aluminosilicates, microglia and micronutrient interactions. EXS 62, 178-189, doi:10.1007/978-3-0348-7460-1_18 (1992).
34 Mittelbronn, M., Dietz, K., Schluesener, H. J. & Meyermann, R. Local distribution of microglia in the normal adult human central nervous system differs by up to one order of magnitude. Acta Neuropathol 101, 249-255, doi:10.1007/s004010000284 (2001).
35 Askew, K. et al. Coupled Proliferation and Apoptosis Maintain the Rapid Turnover of Microglia in the Adult Brain. Cell Rep 18, 391-405, doi:10.1016/j.celrep.2016.12.041 (2017).
36 Bian, Z. et al. Deciphering human macrophage development at single-cell resolution. Nature, doi:10.1038/s41586-020-2316-7 (2020).
37 Evrony, G. D. et al. Single-neuron sequencing analysis of L1 retrotransposition and somatic mutation in the human brain. Cell 151, 483-496, doi:10.1016/j.cell.2012.09.035 (2012).
38 Genovese, G. et al. Clonal hematopoiesis and blood-cancer risk inferred from blood DNA sequence. N Engl J Med 371, 2477-2487, doi:10.1056/NEJMoa1409405 (2014).
39 Jaiswal, S. et al. Age-related clonal hematopoiesis associated with adverse outcomes. N Engl J Med 371, 2488-2498, doi:10.1056/NEJMoa1408617 (2014).
Xi, T., Jones, I. M. & Mohrenweiser, H. W. Many amino acid substitution variants identified in DNA repair genes during human population screenings are predicted to impact protein function. Genomics 83, 970-979, doi:10.1016/j.ygeno.2003.12.016 (2004).
41 Higgins, M. E., Claremont, M., Major, J. E., Sander, C. & Lash, A. E. CancerGenes: a gene selection resource for cancer genome projects. Nucleic Acids Res 35, D721-726, doi:10.1093/nar/gkl811 (2007).
42 Landrum, M. J. et al. ClinVar: public archive of relationships among sequence variation and human phenotype. Nucleic Acids Res 42, D980-985, doi:10.1093/nar/gkt1113 (2014).
43 Chakravarty, D. et al. OncoKB: A Precision Oncology Knowledge Base. JCO Precis Oncol 2017, doi:10.1200/PO.17.00011 (2017).
44 Sando, S. B. et al. APOE epsilon 4 lowers age at onset and is a high risk factor for Alzheimer's disease; a case control study from central Norway. BMC Neurol 8, 9, doi:10.1186/1471-2377-8-9 (2008).
Jaiswal, S. & Libby, P. Clonal haematopoiesis: connecting ageing and inflammation in cardiovascular disease. Nat Rev Cardiol 17, 137-144, doi:10.1038/s41569-019-0247-5 (2020).
46 Liyasova, M. S., Ma, K. & Lipkowitz, S. Molecular pathways: cb1 proteins in tumorigenesis and antitumor immunity-opportunities for cancer treatment. Clin Cancer Res 21, 1789-1794, doi:10.1158/1078-0432.CCR-13-2490 (2015).
47 Aoki, Y. et al. Gain-of-function mutations in RIT1 cause Noonan syndrome, a RAS/MAPK pathway syndrome. Am J Hum Genet 93, 173-180, doi:10.1016/j.ajhg.2013.05.021 (2013).
48 Brand, K., Kentsch, H., Glashoff, C. & Rosenberger, G. RASopathy-associated CBL germline mutations cause aberrant ubiquitylation and trafficking of EGFR. Hum Mutat 35, 1372-1381, doi:10.1002/humu.22682 (2014).
49 Schnittger, S. et al. Molecular analyses of 15,542 patients with suspected BCR-ABL1-negative myeloproliferative disorders allow to develop a stepwise diagnostic workflow. Haematologica 97, 1582-1585, doi:10.3324/haematol.2012.064683 (2012).
50 Dunbar, A. J. et al. 250K single nucleotide polymorphism array karyotyping identifies acquired uniparental disomy and homozygous mutations, including novel missense substitutions of c-Cbl, in myeloid malignancies. Cancer Res 68, 10349-10357, doi:10.1158/0008-5472.CAN-08-2754 (2008).
51 Bernard, V. et al. Applicability of next-generation sequencing to decalcified formalin-fixed and paraffin-embedded chronic myelomonocytic leukaemia samples. Int J Clin Exp Pathol 7, 1667-1676 (2014).
52 Grand, F. H. et al. Frequent CBL mutations associated with 11q acquired uniparental disomy in myeloproliferative neoplasms. Blood 113, 6182-6192, doi:10.1182/blood-2008-12-194548 (2009).
53 Klampfl, T. et al. Complex patterns of chromosome 11 aberrations in myeloid malignancies target CBL, MLL, DDB1 and LMO2. PLoS One 8, e77819, doi:10.1371/journal.pone.0077819 (2013).
54 Piazza, R. et al. Recurrent SETBP1 mutations in atypical chronic myeloid leukemia. Nat Genet 45, 18-24, doi:10.1038/ng.2495 (2013).
55 Niemeyer, C. M. et al. Germline CBL mutations cause developmental abnormalities and predispose to juvenile myelomonocytic leukemia. Nat Genet 42, 794-800, doi:10.1038/ng.641 (2010).
56 Javadi, M., Richmond, T. D., Huang, K. & Barber, D. L. CBL linker region and RING finger mutations lead to enhanced granulocyte-macrophage colony-stimulating factor (GM-CSF) signaling via elevated levels of JAK2 and LYN. J Biol Chem 288, 19459-19470, doi:10.1074/jbc.M113.475087 (2013).
57 Rauen, K. A. The RASopathies. Annu Rev Genomics Hum Genet 14, 355-369, doi:10.1146/annurev-genom-091212-153523 (2013).
58 Tidyman, W. E. & Rauen, K. A. The RASopathies: developmental syndromes of Ras/MAPK pathway dysregulation. Curr Opin Genet Dev 19, 230-236, doi:10.1016/j.gde.2009.04.001 (2009).
59 Roberts, A. E., Allanson, J. E., Tartaglia, M. & Gelb, B. D. Noonan syndrome. Lancet 381, 333-342, doi:10.1016/S0140-6736(12)61023-X (2013).
60 Kang, M. & Lee, Y. S. The impact of RASopathy-associated mutations on CNS development in mice and humans. Mol Brain 12, 96, doi:10.1186/s13041-019-0517-5 (2019).
61 Meyer Zum Buschenfelde, U. et al. RIT1 controls actin dynamics via complex formation with RAC1/CDC42 and PAK1. PLoS Genet 14, e1007370, doi:10.1371/journal.pgen.1007370 (2018).
62 Unger, S. et al. Mutations in the cyclin family member FAM58A cause an X-linked dominant disorder characterized by syndactyly, telecanthus and anogenital and renal malformations. Nat Genet 40, 287-289, doi:10.1038/ng.86 (2008).
63 Guen, V. J. et al. CDK10/cyclin M is a protein kinase that controls ETS2 degradation and is deficient in STAR syndrome. Proc Natl Acad Sci USA 110, 19525-19530, doi:10.1073/pnas.1306814110 (2013).
64 Lachen-Montes, M. et al. An early dysregulation of FAK and MEK/ERK signaling pathways precedes the beta-amyloid deposition in the olfactory bulb of APP/PS1 mouse model of Alzheimer's disease. J Proteomics 148, 149-158, doi:10.1016/j.jprot.2016.07.032 (2016).
65 Song, X., Ma, F. & Herrup, K. Accumulation of Cytoplasmic DNA Due to ATM Deficiency Activates the Microglial Viral Response System with Neurotoxic Consequences. J Neurosci 39, 6378-6394, doi:10.1523/JNEUROSCI.0774-19.2019 (2019).
66 Zannini, L., Delia, D. & Buscemi, G. CHK2 kinase in the DNA damage response and beyond. J Mol Cell Biol 6, 442-457, doi:10.1093/jmcb/mju045 (2014).
67 Drummond, E., & Wisniewski, T. Alzheimer's Disease: Experimental Models and Reality. Acta Neuropathol 133(2), 155-175 (2017).
68 Puzzo, D., et al. Behavioral Assays with Mouse Models fo Alzheimer's Disease: Practical Considerations and Guidelines. Biochem Pharmacol 88(4), 450-467 (2014).
69 Mass, E. et al. A somatic mutation in erythro-myeloid progenitors causes neurodegenerative disease. Nature 549, 389-393, doi:10.1038/nature23672 (2017).
70 Cheng, D. T. et al. Memorial Sloan Kettering-Integrated Mutation Profiling of Actionable Cancer Targets (MSK-IMPACT): A Hybridization Capture-Based Next-Generation Sequencing Clinical Assay for Solid Tumor Molecular Oncology. J Mol Diagn 17, 251-264, doi:10.1016/j.jmoldx.2014.12.006 (2015).
71 Martincorena, I. et al. Tumor evolution. High burden and pervasive positive selection of somatic mutations in normal human skin. Science 348, 880-886, doi:10.1126/science.aaa6806 (2015).
72 Martincorena, I. et al. Somatic mutant clones colonize the human esophagus with age. Science 362, 911-917, doi:10.1126/science.aau3879 (2018).
73 Martincorena, I. & Campbell, P. J. Somatic mutation in cancer and normal cells. Science 349, 1483-1489, doi:10.1126/science.aab4082 (2015).
74 Reiner, A., Yekutieli, D. & Benjamini, Y. Identifying differentially expressed genes using false discovery rate controlling procedures. Bioinformatics 19, 368-375, doi:10.1093/bioinformatics/btf877 (2003).
75 Alexandrov, L. B. et al. Signatures of mutational processes in human cancer. Nature 500, 415-421, doi:10.1038/naturel2477 (2013).
76 Landrum, M. J. et al. ClinVar: public archive of relationships among sequence variation and human phenotype. Nucleic Acids Res 42, D980-985, doi:10.1093/nar/gkt1113 (2014).
77 Chakravarty, D. et al. OncoKB: A Precision Oncology Knowledge Base. JCO Precis Oncol 2017, doi:10.1200/PO.17.00011 (2017).
78 Tidyman, W. E. & Rauen, K. A. The RASopathies: developmental syndromes of Ras/MAPK pathway dysregulation. Curr Opin Genet Dev 19, 230-236, doi:10.1016/j.gde.2009.04.001 (2009).
79 Tidyman, W. E. & Rauen, K. A. Expansion of the RASopathies. Curr Genet Med Rep 4, 57-64, doi:10.1007/s40142-016-0100-7 (2016).
80 Rauen, K. A. The RASopathies. Annu Rev Genomics Hum Genet 14, 355-369, doi:10.1146/annurev-genom-091212-153523 (2013).
81 Galatro, T. F. et al. Transcriptomic analysis of purified human cortical microglia reveals age-associated changes. Nat Neurosci 20, 1162-1171, doi:10.1038/nn.4597 (2017).
82 Gosselin, D. et al. An environment-dependent transcriptional network specifies human microglia identity. Science 356, doi:10.1126/science.aa13222 (2017).
83 Love, M. I., Huber, W. & Anders, S. Moderated estimation of fold change and dispersion for RNA-seq data with DESeq2. Genome Biol 15, 550, doi:10.1186/s13059-014-0550-8 (2014).

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

1. A method for treating or preventing Alzheimer's disease in a subject in need thereof, comprising administering to the subject a therapeutically effective amount of a MAP kinase (MAPK) inhibitor, or a pharmaceutically acceptable salt thereof, wherein the subject is suspected of having, at risk for, or diagnosed as having Alzheimer's disease.

2. A method for delaying the onset of one or more signs or symptoms of Alzheimer's disease in a subject in need thereof, comprising administering to the subject a therapeutically effective amount of a MAP kinase (MAPK) inhibitor, or a pharmaceutically acceptable salt thereof, wherein the subject is suspected of having, at risk for, or diagnosed as having Alzheimer's disease.

3. A method for increasing the survival of a subject in need thereof, comprising administering to the subject a therapeutically effective amount of a MAP kinase (MAPK) inhibitor, or a pharmaceutically acceptable salt thereof, wherein the subject is suspected of having, at risk for, or diagnosed as having Alzheimer's disease.

4. A method for attenuating cognitive decline in a subject in need thereof, comprising administering to the subject a therapeutically effective amount of a MAP kinase (MAPK) inhibitor, or a pharmaceutically acceptable salt thereof, wherein the subject is suspected of having, at risk for, or diagnosed as having Alzheimer's disease.

5. A method for treating or preventing Alzheimer's disease, delaying the onset of one or more signs or symptoms of Alzheimer's disease, increasing the survival of a subject, and/or attenuating cognitive decline in a subject in need thereof, wherein the subject is suspected of having, at risk for, or diagnosed as having Alzheimer's disease, the method comprising:
(a) isolating a tissue sample from the subject;
(b) determining whether the tissue expresses a MAPK-activating mutation; and
(c) administering to the subject a therapeutically effective amount of a MAPK inhibitor, or a pharmaceutically acceptable salt thereof, when the isolated tissue expresses the MAPK-activating mutation.

6. The method of claim 5, wherein the tissue sample is blood or brain tissue.

7. The method of claim 5, wherein the MAPK-activating mutation comprises a mutation in one or more genes selected from CBL, RIT1, BRAF, PTPN11, KRAS, NF1, and FAM58A.

8. The method of claim 7, wherein the mutation in CBL, NF1, or FAM58A is a loss-of-function mutation, and wherein the mutation in RIT1, BRAF, KRAS, or PTPN11 is a gain-of-function mutation.

9. The method of claim 8, wherein:
the CBL loss-of-function mutation comprises one or more of I383M, C404Y, C416S, C384Y, and R420Q;
the NF1 loss-of-function mutation comprises c.7325T>G STOP;
the RIT1 gain-of-function mutation comprises one or more of F99C and M107V;
the BRAF gain-of-function mutation comprises L505H;
the KRAS gain-of-function mutation comprises A59G;
the PTPN11 gain-of-function mutation comprises T731; and
the FAM58A loss-of-function mutation comprises c.423+5A>C.

10.-14. (canceled)

15. The method of claim 1, wherein the subject comprises a MAPK-activating mutation in one or more genes selected from CBL, RIT1, BRAF, PTPN11, KRAS, NF1, and FAM58A.

16. The method of claim 15, wherein the mutation in CBL, NF1, or FAM58A is a loss-of-function mutation, and wherein the mutation in RIT1, BRAF, KRAS, or PTPN11 is a gain-of-function mutation.

17. The method of claim 16, wherein:
the CBL loss-of-function mutation comprises one or more of I383M, C404Y, C416S, C384Y, and R420Q;
the NF1 loss-of-function mutation comprises c.7325T>G STOP;
the RIT1 gain-of-function mutation comprises one or more of F99C and M107V;
the BRAF gain-of-function mutation comprises L505H;
the KRAS gain-of-function mutation comprises A59G;
the PTPN11 gain-of-function mutation comprises T731; and
the FAM58A loss-of-function mutation comprises c.423+5A>C.

18.-23. (canceled)

24. The method of claim 5, wherein the subject carries one APOE4 risk allele.

25. The method of claim 5, wherein the subject is suspected of having, is at risk for, or is diagnosed as having intermediate onset Alzheimer's disease.

26. The method of claim 5, wherein the Alzheimer's disease is characterized by one or more of: cognitive dysfunction or decline; memory loss; agitation; mood swings; impaired judgment; dementia; difficulty with abstract thinking; difficulty with familiar tasks; disorientation; diminished communication skills; repetitive speech or actions; impaired visuospatial abilities; impaired speaking, reading, and writing; withdrawal; depression; loss of recognition; loss of motor skills and sense of touch; delusions; paranoia; verbal or physical aggression; and sleep disorders.

27. The method of claim 5, wherein the MAPK inhibitor is selected from the group consisting of: Gefitinib, Erlotinib, Lapatinib, Sunitinib, Sorafenib, Nilotinib, Dasatinib, Imatinib, Tipifarnib, Sorafenib, U0126, PD184352, AZD6244, BIX02188, BIX02189, SB203580, SB202190, BIRB-796, LY2228820, VX-702, VX-745, and TAK-715.

28. The method of claim 5, wherein the route of administration of the MAPK inhibitor is parenteral, intradermal, intramuscular, intraperitoneal, intravenous, subcutaneous, intranasal, epidural, oral, sublingual, intranasal, intracerebral, intracerebroventricular, intrathecal, intravaginal, transdermal, rectal, by inhalation, or topical.

29. The method of claim 5, further comprising separately, sequentially, or simultaneously administering an additional therapeutic agent to the subject.

30. The method of claim 29, wherein the additional therapeutic agent comprises a BRAF, MEK, and/or CSF-1R inhibitor, or a pharmaceutically acceptable salt thereof.

31. The method of claim 29, wherein the BRAF inhibitor is selected from the group consisting of vemurafenib, dabrafenib, encorafenib, PLX7904, PLX8394, GDC-0879, LGX818, and PLX4720, the MEK inhibitor is selected from the group consisting of AZD8330, refametinib, E6201, MEK162 (binimetinib), PD0325901, pimasertib, R04987655, selumetinib, TAK-733, GDC-0623, WX-544, cobimetinib, and trametinib, and the CSF-1R inhibitor is selected from the group consisting of PLX5622, GW2580, BLZ945, pexidartinib (PLX3397), ARRY-382, PLX7486, and JNJ-40346527.

32.-33. (canceled)


SEQUENCE LISTING (1 sequence)

SEQ ID NO: 1 - protein, 22 aa, synthetic construct
KKKVRSGLYRSPSMPENLNRPR